(19) United States
(12) Patent Application Publication
(10) Pub. No.: US 2019/0256626 A1
(43) Pub. Date: Aug. 22, 2019

(54) SURFACTANTS AND METHODS FOR MAKING SAME

(71) Applicant: REGENTS OF THE UNIVERSITY OF MINNESOTA, Minneapolis, MN (US)

(72) Inventors: Lindsay Michelle JOHNSON, Falls Church, VA (US); Ziang LI, Minneapolis, MN (US); Marc Andrew HILLMYER, Minneapolis, MN (US)

(21) Appl. No.: 16/347,498

(22) PCT Filed: Nov. 11, 2017
(86) PCT No.: PCT/US2017/061129

§ 371 Date: May 3, 2019

Related U.S. Application Data
(60) Provisional application No. 62/420,373, filed on Nov. 10, 2016.

(51) Int. Cl. C08F 20/58; A61K 47/69; A61K 9/107; A61K 47/58; A61K 9/19

(52) U.S. Cl. CPC C08F 20/58; A61K 47/6907; C08F 2438/03; A61K 47/58; A61K 9/19; A61K 9/1075

(57) ABSTRACT

Disclosed herein are surfactants consisting of a hydrophilic segment having repeating units and a first end and a second end. In these surfactants, the first end has a hydrophilic first end group and the second end has a hydrophobic second end group. These surfactants form micelles. Also disclosed herein are methods for making these surfactants and for using them in formulations with biologically active materials.

### CROSS-REFERENCE TO RELATED APPLICATIONS

This application claims the benefit of priority to U.S. Provisional Application No. 62/420,373, filed, Nov. 10, 2016, the disclosure of which is incorporated herein by reference in its entirety.

### BACKGROUND OF THE DISCLOSURE

### Field of the Disclosure

This invention relates to surface active agents, i.e., surfactants. In addition, this invention related to the preparation of such surfactants, and to formulations comprising such surfactants.

### Technical Background

Hydrophobic active pharmaceutical ingredients (APIs) often suffer from poor solubility in aqueous media. While formulations of these APIs with surfactants can increase the effective solubility of the API, most surfactants are either small-molecule surfactants, which perform poorly, or large-molar-mass diblock polymer systems, which require complex synthetic procedures (e.g., two-step sequential polymerization or coupling of two polymers). Furthermore, existing diblock systems are generally troubled by slow release of the API, because the API is required to diffuse from the core of a micelle having a diameter of tens or hundreds of nanometers. Finally, such surfactants are often designed for intravenous injection, which is highly inconvenient, e.g., for APIs that must be administered orally.

Reversible addition-fragmentation chain transfer (RAFT) polymerization is a reversible-deactivation radical polymerization method that makes use of a chain transfer agent (CTA) to afford control over the generated molecular weight and polydispersity during a free-radical polymerization. A RAFT CTA generally has the structure:

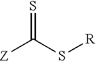

comprising a Z group and an R group bonded to opposite ends of a “RAFT group.” Upon termination of RAFT polymerization, a RAFT group and R group remain bonded to the terminal end of the synthesized polymer. These groups are generally considered undesirable, and much effort has been devoted to methods for cleaving or replacing this terminal RAFT group.

Accordingly, there remains a need for surfactants that can be used to prepare formulations further comprising low-solubility compounds, such as hydrophobic APIs, and provide for rapid release of the low solubility compound at sustained, enhanced concentrations.

### BRIEF DESCRIPTION OF THE FIGURES

FIG. 1 is a set of representative SEM images of (a) Surfactant 1 (without phenytoin), (b) Dispersion 1, (c) Dispersion 2, and (d) Dispersion 13, as described in more detail in Example 3, below.

FIG. 2 is a representative SEM image of crystalline phenytoin, as described in more detail in Example 3, below.

FIG. 3 is a set of representative PXRD spectra of Dispersions 1, 2, 13, 15, 5, and 16, as described in more detail in Example 3, below.

FIG. 4 is a set of dissolution profiles of the Dispersions of Example 4, below.

FIG. 5 is a set of graphs of solid dispersion dissolution vs. supersaturation maintenance as a function of the surfactant, as described in more detail in Example 4, below.

FIG. 6 is a set of graphs of solid dispersion dissolution vs. supersaturation maintenance as a function of the surfactant, as described in more detail in Example 4, below.

FIG. 7 is a set of representative SEM images of Dispersions A, C, E, F, H, J, and pure oxaprozin and 5-methyl-5-phenylhydantoin (5-m-5-p), as described in more detail in Example 5, below.

FIG. 8 is a set of dissolution-time curves for Dispersions A-J and oxaprozin, as described in more detail in Example 5, below.

FIG. 9 is a set of three dissolution-time curves for Dispersions A-L, as described in more detail in Example 6, below.

FIG. 10 is a dissolution-time curve for Dispersions M, G, C, N, O, P, Q, and K, as described in more detail in Example 6, below.

FIG. 11 is a set of representative WAXS spectra of Dispersions M, G, C, N, O, P, Q, and K, and crystalline phenytoin, as described in more detail in Example 6, below.

FIG. 12 is a set of representative SEM images of Dispersions M, K, C, O, and P, and crystalline phenytoin as described in more detail in Example 6, below.

FIG. 13 is a set of representative cryo-TEM images of Dispersion B, as described in more detail in Example 6, below.

FIG. 14 is a set of dissolution profiles of the Dispersions of Example 7, below.

FIG. 15 is a set of dissolution profiles of the Dispersions of Example 7, below.

### SUMMARY OF THE DISCLOSURE

In one aspect, the disclosure provides a surfactant consisting of a hydrophilic segment having repeating units and a first end and a second end;

the first end having a hydrophilic first end group; and

the second end having a second end group; wherein
the first end group comprises from 2 to 20 carbons;
the second end group comprises a linker and a hydrophobic unit, wherein the hydrophobic unit comprises from 2 to 40 carbons; and
the ratio of the molar mass of the hydrophilic segment in kg/mol to the number of carbons in the second end group is from about 0.05 to about 1.

In another aspect, the disclosure provides a surfactant having the formula

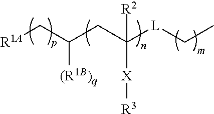

wherein;
n is from 1 to 1000;
m is from 1 to 39;
p is 0, 1, 2, or 3;
q is 0, 1, or 2;
R^{1A} is selected from C₁-C₅fluoroalkyl, C₁-C₅ hydroxyalkyl, (C₁-C₅ alkoxy)C₁-C₃alkyl, —C(O)R^{1C}, —C(S)R^{1C}, —S(O)₁₋₂R^{1C}, —C(O)OR^{1C}, —C(O)NR^{1D}R^{1C}, —C(O)SR^{1C}, —C(S)OR^{1C}, —C(S)NR^{1D}R^{1C}, —C(S)SR^{1C}, —C(NR^{1D})NR^{1D}R^{1C} and —S(O)₁₋₂NR^{1D}R^{1C};
each R^{1B} is independently selected from H, C₁-C₄ alkyl, C₁-C₄ alkenyl, C₁-C₄ alkynyl, C₁-C₅ fluoroalkyl, halogen, nitro, and —CN;
each R² is independently selected from H and methyl;
each X is independently selected from a bond, —O—, and —NR^{1E}—, in which
   each R^{1E} is independently selected from H and methyl;
each R³ is independently selected from —C(O)R^{1C}, —C(O)OR^{1C}, —C(O)NR^{1D}R^{1C}, in which
   each R^{1C} is independently selected from H, C₁-C₄ alkyl, C₁-C₄ alkenyl, C₁-C₄ alkynyl, C₁-C₅ fluoroalkyl, C₁-C₅ hydroxyalkyl and (C₁-C₅ alkoxy)C₁-C₃ alkyl, and
   each R^{1D} is independently selected from H, C₁-C₄ alkyl, C₁-C₄ alkenyl, C₁-C₄ alkynyl, C₁-C₅ fluoroalkyl, C₁-C₅ hydroxyalkyl, (C₁-C₅ alkoxy)C₁-C₃ alkyl, —S(O)₁₋₂(C₁-C₃ alkyl), —C(O)(C₁-C₃ alkyl) and —C(O)O(C₁-C₃ alkyl);
L is selected from

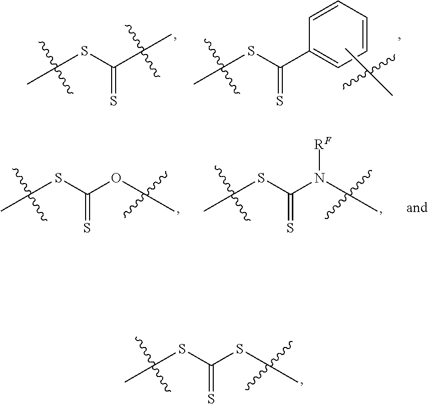

in which
each R^{F} is selected from H and C₁-C₄ alkyl; and
   wherein n and m are selected such that the ratio of the molar mass in kg/mol of the segment of the surfactant having the partial structure

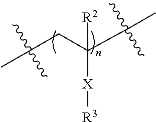

to (m+1) is from about 0.05 to about 10.

In another aspect, the disclosure provides a formulation comprising
a surfactant consisting of a hydrophilic segment having repeating units and a first end and a second end;
   the first end having a hydrophilic first end group; and
   the second end having a second end group; wherein
      the first end group comprises from 2 to 20 carbons;
      the second end group comprises a linker and a hydrophobic unit, wherein the hydrophobic unit comprises from 2 to 40 carbons; and
   the ratio of the molar mass of the hydrophilic segment in kg/mol to the number of carbons in the second end group is from about 0.05 to about 10; or
a surfactant as otherwise described herein or a surfactant synthesized according to a method described herein; and
   hydroxypropyl methylcellulose acetate succinate (HPMCAS).

### DETAILED DESCRIPTION

The particulars shown herein are by way of example and for purposes of illustrative discussion of the preferred embodiments of the invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice. Thus, before the disclosed processes and devices are described, it is to be understood that the aspects described herein are not limited to specific embodiments, apparati, or configurations, and as such can, of course, vary. It is also to be understood that the terminology used herein is for the purpose of describing particular aspects only and, unless specifically defined herein, is not intended to be limiting.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All methods described herein can be performed in any suitable order of steps unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of 0.20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

All percentages, ratios and proportions herein are by weight, unless otherwise specified. A weight percent (weight %, also as wt. %) of a component, unless specifically stated to the contrary, is based on the total weight of the composition in which the component is included (e.g., on the total amount of the reaction mixture).

Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Several embodiments of this invention are described herein. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All patents and printed publications are individually incorporated herein by reference in their entirety.

Terms used herein may be preceded and/or followed by a single dash, “-”, or a double dash, “=”, to indicate the bond order of the bond between the named substituent and its parent moiety; a single dash indicates a single bond and a double dash indicates a double bond or a pair of single bonds in the case of a spiro-substituent. In the absence of a single or double dash it is understood that a single bond is formed between the substituent and its parent moiety; further, substituents are intended to be read “left to right” with reference to the chemical structure referred to unless a dash indicates otherwise. For example, arylalkyl, arylalkyl-, and -alkylaryl indicate the same functionality.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety can refer to a monovalent radical (e.g. CH₃—CH₂—), in some circumstances a bivalent linking moiety can be “alkyl,” in which case those skilled in the art will understand the alkyl to be a divalent radical (e.g., —CH₂CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for O, and 2, 4, or 6 for S, depending on the oxidation state of the S). Nitrogens in the presently disclosed compounds can be hypervalent, e.g., an N-oxide or tetrasubstituted ammonium salt. On occasion a moiety may be defined, for example, as -B-(A)ₐ, wherein a is 0 or 1. In such instances, when a is 0 the moiety is —B and when a is 1 the moiety is -B-A.

As used herein, the term “alkyl” includes a saturated hydrocarbon having a designed number of carbon atoms, such as 1 to 40 carbons (i.e., inclusive of 1 and 40), 1 to 35 carbons, 1 to 25 carbons, 1 to 20 carbons, or 1, 2, 3, 4, 5, 6, 7, 8, 9, 10, 11, 12, 13, 14, 15, 16, 17, or 18. Alkyl group may be straight or branched and depending on context, may be a monovalent radical or a divalent radical (i.e., an alkylene group). For example, the moiety “—(C₁-C₆alkyl)-O—” signifies connection of an oxygen through an alkylene bridge having from 1 to 6 carbons and C₁-C₃alkyl represents methyl, ethyl, and propyl moieties. Examples of “alkyl” include, for example, methyl, ethyl, propyl, isopropyl, butyl, iso-, sec- and tert-butyl, pentyl, and hexyl.

The term “alkoxy” represents an alkyl group of indicated number of carbon atoms attached to the parent molecular moiety through an oxygen bridge. Examples of “alkoxy” include, for example, methoxy, ethoxy, propoxy, and isopropoxy.

The term “alkenyl” as used herein, unsaturated hydrocarbon containing from 2 to 10 carbons (i.e., inclusive of 2 and 10), 2 to 8 carbons, 2 to 6 carbons, or 2, 3, 4, 5 or 6, unless otherwise specified, and containing at least one carbon-carbon double bond. Alkenyl group may be straight or branched and depending on context, may be a monovalent radical or a divalent radical (i.e., an alkenylene group). For example, the moiety “—(C₂-C₆ alkenyl)-O—” signifies connection of an oxygen through an alkenylene bridge having from 2 to 6 carbons. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkynyl” as used herein, unsaturated hydrocarbon containing from 2 to 10 carbons (i.e., inclusive of 2 and 10), 2 to 8 carbons, 2 to 6 carbons, or 2, 3, 4, 5 or 6 unless otherwise specified, and containing at least one carbon-carbon triple bond. Alkynyl group may be straight or branched and depending on context, may be a monovalent radical or a divalent radical (i.e., an alkynylene group). For example, the moiety “—(C₂-C₆ alkynyl)-O—” signifies connection of an oxygen through an alkynylene bridge having from 2 to 6 carbons. Representative examples of alkynyl include, but are not limited to, acetylenyl, 1-propynyl, 2-propynyl, 3-butynyl, 2-pentynyl, and 1-butynyl.

The term “aryl” represents an aromatic ring system having a single ring (e.g., phenyl) which is optionally fused to other aromatic hydrocarbon rings or non-aromatic hydrocarbon or heterocyclic rings. “Aryl” includes ring systems having multiple condensed rings and in which at least one is carbocyclic and aromatic, (e.g., 1,2,3,4-tetrahydronaphthyl, naphthyl). Examples of aryl groups include phenyl, 1-naphthyl, 2-naphthyl, indanyl, indenyl, dihydronaphthyl, fluorenyl, tetralinyl, and 6,7,8,9-tetrahydro-5H-benzo[a]cycloheptenyl. “Aryl” also includes ring systems having a first carbocyclic, aromatic ring fused to a nonaromatic heterocycle, for example, 1H-2,3-dihydrobenzofuranyl and tetrahydroisoquinolinyl. The aryl groups herein are unsubstituted or, when specified as “optionally substituted”, can unless stated otherwise be substituted in one or more substitutable positions with various groups as indicated.

The term “heteroaryl” refers to an aromatic ring system containing at least one aromatic heteroatom selected from nitrogen, oxygen and sulfur in an aromatic ring. Most commonly, the heteroaryl groups will have 1, 2, 3, or 4 heteroatoms. The heteroaryl may be fused to one or more non-aromatic rings, for example, cycloalkyl or heterocycloalkyl rings, wherein the cycloalkyl and heterocycloalkyl rings are described herein. In one embodiment of the present compounds the heteroaryl group is bonded to the remainder of the structure through an atom in a heteroaryl group aromatic ring. In another embodiment, the heteroaryl group is bonded to the remainder of the structure through a non-aromatic ring atom. Examples of heteroaryl groups include, for example, pyridyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, benzo[1,4]oxazinyl, triazolyl, tetrazolyl, isothiazolyl, naphthyridinyl, isochromanyl, chromanyl, isoindolinyl, isobenzothienyl, benzoxazolyl, pyridopyridinyl, purinyl, benzodioxolyl, triazinyl, pteridinyl, benzothiazolyl, imidazopyridinyl, imidazothiazolyl, benzisoxazinyl, benzoxazinyl, benzopyranyl, benzothiopyranyl, chromonyl, chromanonyl, pyridinyl-N-oxide, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. Preferred heteroaryl groups include pyridyl, pyrimidyl, quinolinyl, indolyl, pyrrolyl, furanyl, thienyl and imidazolyl, pyrazolyl, indazolyl, thiazolyl and benzothiazolyl. In certain embodiments, each heteroaryl is selected from pyridyl, pyrimidinyl, pyridazinyl, pyrazinyl, imidazolyl, isoxazolyl, pyrazolyl, oxazolyl, thiazolyl, furanyl, thienyl, pyrrolyl, oxadiazolyl, thiadiazolyl, triazolyl, tetrazolyl, isothiazolyl, pyridinyl-N-oxide, pyrrolyl N-oxide, pyrimidinyl N-oxide, pyridazinyl N-oxide, pyrazinyl N-oxide, imidazolyl N-oxide, isoxazolyl N-oxide, oxazolyl N-oxide, thiazolyl N-oxide, pyrrolyl N-oxide, oxadiazolyl N-oxide, thiadiazolyl N-oxide, triazolyl N-oxide, and tetrazolyl N-oxide. Preferred heteroaryl groups include pyridyl, pyrimidyl, quinolinyl, indolyl, pyrrolyl, furanyl, thienyl, imidazolyl, pyrazolyl, indazolyl, thiazolyl and benzothiazolyl. The heteroaryl groups herein are unsubstituted or, when specified as “optionally substituted”, can unless stated otherwise be substituted in one or more substitutable positions with various groups, as indicated.

The term “heterocycloalkyl” refers to a non-aromatic ring or ring system containing at least one heteroatom that is preferably selected from nitrogen, oxygen and sulfur, wherein said heteroatom is in a non-aromatic ring. The heterocycloalkyl may have 1, 2, 3 or 4 heteroatoms. The heterocycloalkyl may be saturated (i.e., a heterocycloalkyl) or partially unsaturated (i.e., a heterocycloalkenyl). Heterocycloalkyl includes monocyclic groups of three to eight annular atoms as well as bicyclic and polycyclic ring systems, including bridged and fused systems, wherein each ring includes three to eight annular atoms. The heterocycloalkyl ring is optionally fused to other heterocycloalkyl rings and/or non-aromatic hydrocarbon rings. In certain embodiments, the heterocycloalkyl groups have from 3 to 7 members in a single ring. In other embodiments, heterocycloalkyl groups have 5 or 6 members in a single ring. In some embodiments, the heterocycloalkyl groups have 3, 4, 5, 6 or 7 members in a single ring. Examples of heterocycloalkyl groups include, for example, azabicyclo[2.2.2]octyl (in each case also “quinuclidinyl” or a quinuclidine derivative), azabicyclo[3.2.1]octyl, 2.5-diazabicyclo[2.2.1]heptyl, morpholinyl, thiomorpholinyl, thiomorpholinyl S-oxide, thiomorpholinyl S,S-dioxide, 2-oxazolidonyl, piperazinyl, homopiperazinyl, piperazinonyl, pyrrolidinyl, azepanyl, azetidinyl, pyrrolinyl, tetrahydropyranyl, piperidinyl, tetrahydrofuranyl, tetrahydrothienyl, 3,4-dihydroisoquinolin-2(1H)-yl, isoindolindionyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, imidazolidonyl, tetrahydrothienyl S-oxide, tetrahydrothienyl S,S-dioxide and homothiomorpholinyl S-oxide. Especially desirable heterocycloalkyl groups include morpholinyl, 3,4-dihydroisoquinolin-2(1H)-yl, tetrahydropyranyl, piperidinyl, aza-bicyclo[2.2.2]octyl, γ-butyrolactonyl (i.e., an oxo-substituted tetrahydrofuranyl), γ-butryolactamyl (i.e., an oxo-substituted pyrrolidine), pyrrolidinyl, piperazinyl, azepanyl, azetidinyl, thiomorpholinyl, thiomorpholinyl S,S-dioxide, 2-oxazolidonyl, imidazolidonyl, isoindolindionyl, piperazinonyl. The heterocycloalkyl groups herein are unsubstituted or, when specified as “optionally substituted”, can unless stated otherwise be substituted in one or more substitutable positions with various groups, as indicated.

The term “cycloalkyl” refers to a non-aromatic carbocyclic ring or ring system, which may be saturated (i.e., a cycloalkyl) or partially unsaturated (i.e., a cycloalkenyl). The cycloalkyl ring optionally fused to or otherwise attached (e.g., bridged systems) to other cycloalkyl rings. Certain examples of cycloalkyl groups present in the disclosed compounds have from 3 to 7 members in a single ring, such as having 5 or 6 members in a single ring. In some embodiments, the cycloalkyl groups have 3, 4, 5, 6 or 7 members in a single ring. Examples of cycloalkyl groups include, for example, cyclohexyl, cyclopentyl, cyclobutyl, cyclopropyl, tetrahydronaphthyl and bicyclo[2.2.1]heptane. The cycloalkyl groups herein are unsubstituted or, when specified as “optionally substituted”, may be substituted in one or more substitutable positions with various groups, as indicated.

The term “ring system” encompasses monocycles, as well as fused and/or bridged polycycles.

The terms “halogen” or “halo” indicate fluorine, chlorine, bromine, and iodine. In certain embodiments of each and every embodiment described herein, the term “halogen” or “halo” refers to fluorine or chlorine. In certain embodiments of each and every embodiment described herein, the term “halogen” or “halo” refers to fluorine.

The term “substituted,” when used to modify a specified group or radical, means that one or more hydrogen atoms of the specified group or radical are each, independently of one another, replaced with the same or different substituent groups as defined below, unless specified otherwise.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

In various aspects and embodiments, the disclosure relates to micelle-forming compounds. The disclosure demonstrates such compounds, which can be made in a single step, to rapidly release and to enhance the solubility of biologically active compounds having low water solubility.

One aspect of the disclosure is a surfactant consisting of a hydrophilic segment having repeating units and a first end and a second end. The first end of the surfactant includes a hydrophilic first end group. As used herein, a “hydrophilic” segment or group is one that, taken independently, would be soluble in polar media, e.g., methanol, water, and the like. In some embodiments, the hydrophilic segment, taken independently, has a solubility in polar media of at least about 1 wt. %, e.g., at least about 2 wt. %, or at least about 3 wt. %, or at least about 4 wt. %, or at least about 5 wt. %, or at least about 6 wt. %, or at least about 7 wt. %, or at least about 8 wt. %, or at least about 9 wt. %, or at least about 10 wt. %. In the materials and methods described herein, the segment and first end group are sufficiently hydrophilic such that the surfactant is soluble in polar media. The person of ordinary skill in the art will appreciate that the hydrophilic segment may be thermoresponsive, provided the segment is sufficiently hydrophilic, e.g., below a lower critical solution temperature (LCST). In some embodiments of the materials and methods as otherwise described herein, the surfactant has a solubility in polar media of at least 1 wt. %, e.g., at least 2 wt. %, or at least 3 wt. %, or at least 4 wt. %, or at least 5 wt. %, or at least 6 wt. %, or at least 7 wt. %, or at least 8 wt. %, or at least 9 wt. %, or at least 10 wt. %.

In the materials and methods described herein, the first end group comprises from 2 to 20 carbons, e.g., from 2 to 19 carbons, or from 2 to 18 carbons, or from 2 to 17 carbons, or from 2 to 16 carbons, or from 2 to 15 carbons, or from 2 to 14 carbons, or from 2 to 13 carbons, or from 2 to 12 carbons, or from 2 to 11 carbons, or from 2 to 10 carbons, or from 2 to 9 carbons, or from 2 to 8 carbons, or from 2 to 7 carbons.

In the materials and methods described herein, the second end of the surfactant includes a linker bound to a hydrophobic unit. As used herein, a “hydrophobic” unit is one that, taken independently, would be poorly soluble or insoluble in polar media, e.g., methanol, water, and the like. In some embodiments, the hydrophobic unit of the surfactant may be any unit capable of spontaneously associating with the hydrophobic unit of another surfactant as otherwise described herein. In some embodiments, the hydrophobic unit, taken independently, has a solubility in polar media of less than about 1 wt. %, or less than about 0.75 wt. %, or less than about 0.5 wt. %, or less than about 0.25 wt. %, or less than about 0.2 wt. %, or less than about 0.1 wt. %, or less than about 0.02 wt. %, or less than about 0.01 wt. %. The hydrophobic unit comprises from 2 to 40 carbons, e.g., from 3 to 40, or from 4 to 40, or from 5 to 40 carbons. In some embodiments, the hydrophobic unit comprises from 6 to 40 carbons, e.g., from 6 to 35, or from 6 to 30, or from 6 to 25, or from 6 to 24, or from 6 to 23, or from 6 to 22, or from 6 to 21, or from 6 to 20, or from 7 to 40, or from 8 to 40, or from 9 to 40, or from 10 to 40, or from 11 to 40, or from 12 to 40, or from 13 to 40, or from 14 to 40, or from 15 to 40, or from 16 to 40, or from 17 to 40, or from 18 to 40, or from 19 to 40, or from 20 to 40, or from 25 to 40, or from 7 to 35, or from 8 to 30, or from 9 to 25, or from 10 to 20, or the number of carbons is 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30 carbons.

In the materials and methods described herein, the ratio of the molar mass of the hydrophilic segment in kg/mol to the number of carbons in the second end group is from about 0.05 to about 10, e.g., from about 0.05 to about 9.5, or from about 0.05 to about 9, or from about 0.05 to about 8.5, or from about 0.05 to about 8, or from about 0.05 to about 7.5, or from about 0.05 to about 7, or from about 0.05 to about 6.5, or from about 0.05 to about 6, or from about 0.05 to about 5.5, or from about 0.05 to about 5, or from about 0.05 to about 4.5, or from about 0.05 to about 4.0, or from about 0.05 to about 3.75, or from about 0.05 to about 3.5, or from about 0.05 to about 3.25, or from about 0.05 to about 3, or from about 0.05 to about 2.75, or from about 0.05 to about 2.5, or from about 0.05 to about 2.25, or from about 0.05 to about 2, or from about 0.05 to about 1.75, or from about 0.05 to about 1.5, or from about 0.05 to about 1.25.

In some embodiments of the materials and methods as otherwise described herein, the ratio of the molar mass of the hydrophilic segment in kg/mol to the number of carbons in the second end group may be from about 0.05 to about 1, e.g., from about 0.05 to about 0.95, or from about 0.05 to about 0.9, or from about 0.05 to about 0.85, or from about 0.05 to about 0.8, or from about 0.05 to about 0.75, or from about 0.05 to about 0.7, or from about 0.05 to about 0.65, or from about 0.05 to about 0.6, or from about 0.05 to about 0.55, or from about 0.05 to about 0.5, or from about 0.05 to about 0.45, or from about 0.05 to about 0.40, or from about 0.05 to about 0.375, or from about 0.05 to about 0.35, or from about 0.05 to about 0.325, or from about 0.05 to about 0.3, or from about 0.05 to about 0.275, or from about 0.05 to about 0.25, or from about 0.05 to about 0.225, or from about 0.05 to about 0.2, or from about 0.05 to about 0.175, or from about 0.05 to about 0.15, or from about 0.05 to about 0.125.

In some embodiments of the materials and methods as otherwise described herein, the linker may be a bond between the hydrophilic segment and the hydrophobic unit, e.g., a C—C bond. In other embodiments, the linker is a group to which the hydrophilic segment and hydrophobic unit are bonded. In some embodiments, the linker may be a chain transfer agent (CTA) group, e.g., 3-mercaptopropionate, benzenethiol, mercaptan, etc. In some embodiments, the CTA group is a reversible addition-fragmentation chain-transfer polymerization (RAFT) CTA group, e.g., dithiobenzoate, dithioester, dithiocarbamate, trithiocarbonate, xanthate, etc. In one example, the hydrophilic segment and hydrophobic segment are each bonded to a sulfur atom of a trithiocarbonate. In another example, the hydrophilic segment and hydrophobic unit are bonded to a sulfur atom and the aryl ring of a dithiobenzoate, respectively.

In some embodiments of the materials and methods as otherwise described herein, the hydrophilic segment may have a molar mass of less than about 20 kg/mol, e.g., less than about 19 kg/mol, or less then about 18 kg/mol, or less than about 17 kg/mol, or less than about 16 kg/mol, or less than about 15 kg/mol, or less than about 14 kg/mol, or less than about 13 kg/mol, or less than about 12 kg/mol, or less than about 11 kg/mol, or less than about 10 kg/mol, or less than about 9 kg/mol, or less than about 8 kg/mol, or less than about 7 kg/mol, or less than about 6 kg/mol, or less than about 5 kg/mol, or less than about 4 kg/mol, or less than about 3 kg/mol, or less than about 2 kg/mol.

In some embodiments of the materials and methods as otherwise described herein, the number of repeating units in the hydrophilic segment is from about 2 to about 1000, e.g., about 2 to about 900, or about 2 to about 800, or about 2 to about 700, or about 2 to about 600, or about 2 to about 500, or about 2 to about 400, or about 2 to about 300, or about 25 to about 900, or about 75 to about 800, or about 100 to about 700, or about 125 to about 600, or about 150 to about 500.

In some embodiments of the materials and methods as otherwise described herein, the number of repeating units in the hydrophilic segment is from about 2 to about 200, e.g., about 2 to about 190, or about 2 to about 180, or about 2 to about 170, or about 2 to about 160, or about 2 to about 150, or at about 2 to about 140, or about 2 to about 130, or about 2 to about 120, or about 2 to about 110, or about 2 to about 100, or about 2 to about 90, or about 2 to about 80, or about 3 to about 180, or about 4 to about 160, or about 5 to about 140, or about 6 to about 130, or about 7 to about 120, or about 8 to about 110, or about 9 to about 100, or about 5 to about 200, or about 10 to about 200, or about 25 to about 200, or about 50 to about 200, or about 75 to about 200, or about 100 to about 200, or the number is about 5, or about 6, or about 7, or about 8, or about 9, or about 10, or about 11, or about 12, or about 13, or about 14, or about 15, or about 16, or about 17, or about 18, or about 19, or about 20, or about 25, or about 30, or about 35, or about 40, or about 45, or about 50, or about 55, or about 60, or about 65, or about 70, or about 75, or about 100, or about 125, or about 150, or about 175.

The person of ordinary skill in the art will appreciate that the repeating units of the hydrophilic segment may be monomers (i.e., the hydrophilic segment may be a polymer). In some embodiments, the repeating units may be different. In certain such embodiments, the repeating units may be two or more different monomers arranged in an alternating manner (i.e., an alternating copolymer), a periodic manner (i.e., a periodic copolymer), or according to a statistical rule (i.e., a statistical copolymer). The person of ordinary skill in the art will appreciate that each repeating unit need not be hydrophilic, provided the segment as a whole is sufficiently hydrophilic (e.g, an arrangement of hydrophobic unit A and hydrophilic unit B of B-B-A-B-B-A), and not amphiphilic (e.g., an arrangement of hydrophobic unit A and hydrophilic unit B of A-A-A-B-B-B). In some embodiments, the repeating units are the same. In certain such embodiments, the repeating units may be the same monomer (i.e., a homopolymer).

In some embodiments of the materials and methods as otherwise described herein, the repeating units of the hydrophilic segment may be one or more hydrophilic monomers, e.g., acrylates, methacrylates, acrylamides, methacrylamides, vinyl esters, vinyl amides, and the like. In some embodiments, the repeating unit may include an N-isopropylamide, an N-dimethylacrylamide, or an N-hydroxyethylacrylamide. In one example, the hydrophilic segment is a homopolymer derived from N-isopropylacrylamide monomers, N-dmiethylacrylamide monomers, or N-hydroxyethylacrylamide monomers. The person of ordinary skill in the art will appreciate that one or more of the repeating units of the hydrophilic segment may be modified, e.g., by side chain modification, provided the segment as a whole is sufficiently hydrophilic. Any such modifications to the hydrophilic segment are considered part of the hydrophilic segment, and would contribute to the molar mass of the hydrophilic segment.

In some embodiments of the materials and methods as otherwise described herein, the repeating unit has the partial structure

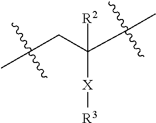

wherein each R² is independently selected from H and methyl; each X is independently selected from a bond, —O—, and —NR^{1E}—, in which each R^{1E} is selected from H and methyl; each R³ is independently selected from —C(O)R^{1C}, —C(O)OR^{1C}, —C(O)NR^{1D}R^{1C}, in which each R^{1C} is independently selected from H, C₁-C₄ alkyl, C₁-C₄ alkenyl, C₁-C₄ alkynyl, C₁-C₅ fluoroalkyl, C₁-C₅ hydroxyalkyl and (C₁-C₅ alkoxy)C₁-C₃ alkyl, and each R^{1D} is independently selected from H, C₁-C₄ alkyl, C₁-C₄ alkenyl, C₁-C₄ alkynyl, C₁-C₅ fluoroalkyl, C₁-C₅ hydroxyalkyl, (C₁-C₅ alkoxy)C₁-C₃ alkyl, —S(O)₁₋₂(C₁-C₃ alkyl), —C(O)(C₁-C₃ alkyl) and —C(O)O(C₁-C₃ alkyl).

In some embodiments of the materials and methods as otherwise described herein, the hydrophobic unit of the second end group may be alkyl, e.g., hexyl, heptyl, octyl, nonyl, decyl, undecyl, dodecyl, tridecyl, tetradecyl, pentadecyl, hexadecyl, heptadecyl, octadecyl, etc. In certain such embodiments, the hydrophobic unit may be linear or branched alkyl, and may be saturated or unsaturated alkyl (i.e., alkenyl or alkynyl). In some embodiments, the hydrophobic unit may be an ether or polyether, e.g., methoxypentyl, poly(ethylene glycol), etc. In some embodiments, the hydrophobic unit may include one or more optionally substituted aryl groups, e.g., polyphenyl ether, poly(p-phenylene vinylene), hexylphenyl, etc. In some embodiments, the hydrophobic unit may include one or more optionally substituted heteroaryl, cycloalkyl, or heterocycloalkyl groups, e.g., pyridyl, indolyl, polypyrrole, cyclohexyl, poly(cyclohexene oxide), piperidinyl, thianyl, pyrrolidinyl, etc. In some embodiments, the hydrophobic unit may be an ester or polyester, e.g., hexyl ethanoate, poly(ethylene succinate), etc. The person of ordinary skill in the art will appreciate that a variety of additional, suitable hydrophobic units are known in the art.

In some embodiments of the materials and methods as otherwise described herein, the first end group includes an alcohol, e.g., pentanol, hexanol, hexanediol, etc. In certain such embodiments, the first end group includes an alcohol and further includes a nitrile, e.g., 5-hydroxy-2-methylpentanenitrile. In some embodiments, the first end group includes a carboxylic acid, e.g., acetic acid, propionic acid, isobutyric acid, etc. In certain such embodiments, the first end group includes a carboxylic acid and further includes a nitrile, e.g., 4-cyanopentanoic acid. In some embodiments, the first end group includes an ester, halide, amine, or anhydride.

In some embodiments of the materials and methods as otherwise described herein, the first end group includes R^{1A}, wherein R^{1A} is selected from C₁-C₅fluoroalkyl, C₁-C₅ hydroxyalkyl, (C₁-C₅ alkoxy)C₁-C₃alkyl, —C(O)R^{1C}, —C(S)R^{1C}, —S(O)₁₋₂R^{1C}, —C(O)OR^{1C}, —C(O)NR^{1D}R^{1C}, —C(O)SR^{1C}, —C(S)OR^{1C}, —C(S)NR^{1D}R^{1C}, —C(S)SR^{1C}, —C(NR^{1D})NR^{1D}R^{1C} and —S(O)₁₋₂NR^{1D}R^{1C}. In certain such embodiments, the first end group includes R^{1a} and further includes one or more R^{1B}, wherein R^{1B} is selected from H, C₁-C₄ alkyl, C₁-C₄ alkenyl, C₁-C₄ alkynyl, C₁-C₅ fluoroalkyl, halogen, nitro, and —CN.

Another aspect of the disclosure is a surfactant having Formula I:

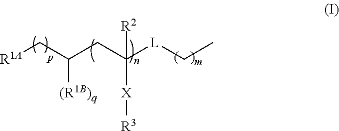

wherein:
n is from 1 to 1000;
m is from 1 to 39;
p is 0, 1, 2, or 3;
q is 0, 1, or 2;
R^{1A} is selected from C₁-C₅fluoroalkyl, C₁-C₅ hydroxyalkyl, (C₁-C₅ alkoxy)C₁-C₃alkyl, —C(O)R^{1C}, —C(S)R^{1C}, —S(O)₁₋₂R^{1C}, —C(O)OR^{1C}, —C(O)NR^{1D}R^{1C}, —C(O)SR^{1C}, —C(S)OR^{1C}, —C(S)NR^{1D}R^{1C}, —C(S)SR^{1C}, —C(NR^{1D})NR^{1D}R^{1C} and —S(O)₁₋₂NR^{1D}R^{1C};
each R^{1B} is selected from H, C₁-C₄ alkyl, C₁-C₄ alkenyl, C₁-C₄ alkynyl, C₁-C₅ fluoroalkyl, halogen, nitro, and —CN;
each R² is independently selected from H and methyl;
each X is independently selected from a bond, —O—, and —NR^{1E}—, in which
   each R^{1E} is selected from H and methyl;
each R³ is independently selected from —C(O)R^{1C}, —C(O)OR^{1C}, —C(O)NR^{1D}R^{1C}, in which
   each R^{1C} is independently selected from H, C₁-C₄ alkyl, C₁-C₄ alkenyl, C₁-C₄ alkynyl, C₁-C₅ fluoroalkyl, C₁-C₅ hydroxyalkyl and (C₁-C₅ alkoxy)C₁-C₃ alkyl, and
   each R^{1D} is independently selected from H, C₁-C₄ alkyl, C₁-C₄ alkenyl, C₁-C₄ alkynyl, C₁-C₅ fluoroalkyl, C₁-C₅ hydroxyalkyl, (C₁-C₅ alkoxy)C₁-C₃ alkyl, —S(O)₁₋₂(C₁-C₃ alkyl), —C(O)(C₁-C₃ alkyl) and —C(O)O(C₁-C₃ alkyl);
L is selected from

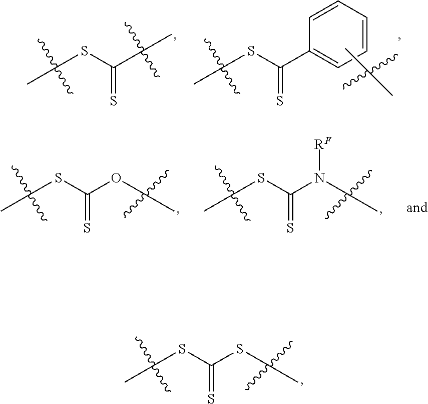

in which
each R^{F} is selected from H and C₁-C₄ alkyl; and
   wherein n and m are selected such that the ratio of the molar mass in kg/mol of the segment of the surfactant having the partial structure

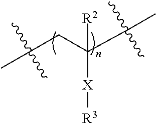

to (m+1) is from about 0.05 to about 10.

In some embodiments of the surfactant having Formula I, n is from 2 to 1000, e.g., from 3 to 1000, or from 4 to 1000, or from 5 to 1000, or from 6 to 1000, or from 7 to 1000, or from 8 to 1000, or from 9 to 1000, or from 10 to 1000, or from 11 to 1000, or from 12 to 1000, or from 13 to 1000, or from 14 to 1000, or from 15 to 1000, or from 20 to 1000, or from 25 to 1000, or from 30 to 1000, or from 35 to 1000, or from 40 to 1000, or from 45 to 1000, or from 50 to 1000, or from 60 to 1000, or from 70 to 1000, or from 80 to 1000, or from 90 to 1000, or from 100 to 1000.

In embodiments of the surfactant having Formula 1, m is from 1 to 39, e.g., from 2 to 39, or from 3 to 39, or from 4 to 39, or from 5 to 39, or from 6 to 39.

In some embodiments of the surfactant having Formula I, m is from 7 to 39, e.g., from 8 to 39, or from 9 to 39, or from 10 to 39, or from 11 to 39, or from 12 to 39, or from 13 to 39, or from 14 to 39, or from 15 to 39, or from 16 to 39, or from 17 to 39.

In some embodiments of the surfactant having Formula I, R^{1A} is selected from C₁-C₅ hydroxyalkyl and —C(O)OR^{1C}, wherein R^{1C} is H. In some embodiments, q is 0. In some embodiments, q is 1 and R^{1B} is selected from C₁-C₄ alkyl and —CN. In some embodiments, q is 2 and each R^{1B} is independently selected from C₁-C₄ alkyl and —CN.

In some embodiments of the surfactant having Formula I, X is a bond. In certain such embodiments, X is a bond and R³ is —C(O)NR^{1D}R^{1C}, in which each R^{1C} is independently selected from H and C₁-C₄ alkyl and each R^{1D} is independently selected from H and C₁-C₄ alkyl.

In some embodiments of the surfactant having Formula I, n and m are selected such that the ratio of the molar mass in kg/mol of the segment of the surfactant having the partial structure

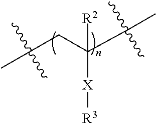

to (m+1) is from about 0.05 to about 9.5, e.g., from about 0.05 to about 9, or from about 0.05 to about 8.5, or from about 0.05 to about 8, or from about 0.05 to about 7.5, or from about 0.05 to about 7, or from about 0.05 to about 6.5, or from about 0.05 to about 6, or from about 0.05 to about 5.5, or from about 0.05 to about 5, or from about 0.05 to about 4.5, or from about 0.05 to about 4, or from about 0.05 to about 3.75, or from about 0.05 to about 3.5, or from about 0.05 to about 3.25, or from about 0.05 to about 3, or from about 0.05 to about 2.75, or from about 0.05 to about 2.5, or from about 0.05 to about 2.25, or from about 0.05 to about 2, or from about 0.05 to about 1.75, or from about 0.05 to about 1.5, or from about 0.05 to about 1.25.

In some embodiments of the surfactant having Formula I, n and m are selected such that the ratio is from about 0.05 to about 0.95, e.g., from about 0.05 to about 0.9, or from about 0.05 to about 0.85, or from about 0.05 to about 0.8, or from about 0.05 to about 0.75, or from about 0.05 to about 0.7, or from about 0.05 to about 0.65, or from about 0.05 to about 0.6, or from about 0.05 to about 0.55, or from about 0.05 to about 0.5, or from about 0.05 to about 0.45, or from about 0.05 to about 0.40, or from about 0.05 to about 0.375, or from about 0.05 to about 0.35, or from about 0.05 to about 0.325, or from about 0.05 to about 0.3, or from about 0.05 to about 0.275, or from about 0.05 to about 0.25, or from about 0.05 to about 0.225, or from about 0.05 to about 0.2, or from about 0.05 to about 0.175, or from about 0.05 to about 0.15, or from about 0.05 to about 0.125.

Another aspect of the disclosure is a mixture of surfactants as otherwise described herein, wherein the dispersity (0) of the mixture is less than about 2.5, e.g., less than about 2.4, or less than about 2.3, or less than about 2.2, or less than about 2.1, or less than about 2.0, or less than about 1.95, or less than about 1.9, or less than about 1.85, or less than about 1.8, or less than about 1.75, or less than about 1.7, or less than about 1.65, or less than about 1.6, or less than about 1.55, or less than about 1.5, or less than about 1.45, or less than about 1.4, or less than about 1.35, or less than about 1.3, or less than about 1.25, or less than about 1.2.

Another aspect of the disclosure is a mixture two or more surfactants as otherwise described herein, for example, a mixture of two surfactants present in a weight ratio within the range of about 90:0 to about 0:90, or about 72:18 to about 18:72, or about 63:27 to about 27:63, or about 54:36 to about 36:54.

Another aspect of the disclosure is a method for preparing a formulation, comprising providing a biologically active compound having low water solubility and combining the biologically active compound with a surfactant or a mixture of surfactants as otherwise described herein. In some embodiments, the biologically active compound has a solubility in water of less than about 5 wt. %, e.g., less than about 4 wt. %, or less then about 3 wt. %, or less than about 2 wt. %, or less than about 1 wt. %, or less than about 0.9 wt. %, or less than about 0.8 wt. %, or less than about 0.7 wt. %, or less than about 0.6 wt. %, or less than about 0.5 wt. %, or less than about 0.4 wt. %, or less than about 0.3 wt. %, or less than about 0.1 wt. %, or less than about 0.075 wt. %, or less than about 0.05 wt. %.

In some embodiments, the biologically active compound may be a compound for treating or preventing diseases and disorders in an animal (including humans and other primates, horses, cattle, swine, and domestic animals such as dogs and cats), e.g., phenytoin, oxaprozin, 5-methyl-5-phenylhydantoin, etc. In other embodiments, the biologically active compound may be a compound for treating plants, e.g., an insecticide, growth hormone, etc.

Another aspect of the disclosure is a formulation comprising a biologically active compound having low water solubility and a surfactant or mixture of surfactants as otherwise described herein. In some embodiments, the ratio of biologically active compound to the surfactant or mixture is from about 1:99 to about 50:50, e.g., from about 1:99 to about 40:60, or from about 1:99 to about 30:70, or from about 1:99 to about 20:80, or from about 1:99 to about 15:85, or from about 1:99 to about 10:90, or the ratio is about 1:99, or about 5:95, or about 10:90, or about 15:85, or about 20:80, or about 25:75, or about 30:70, or about 35:65, or about 40:60, or about 45:55, or about 50:50, based on weight (i.e., wt. %).

Another aspect of the disclosure is a micellar composition comprising a biologically active compound having low water solubility and a surfactant or a mixture of surfactants as otherwise described herein. As used herein, the term “micellar” describes an aggregate of surfactants wherein a hydrophilic region of the surfactants is in contact with the surrounding solvent, and a hydrophobic region of the surfactants is sequestered in or near the center of the aggregate. In some embodiments, the biologically active compound is localized in or near the center of the aggregates of the micellar composition. In some embodiments, the biologically active compound is localized on or near the exterior (i.e., the corona) of the aggregates of the micellar composition.

Another aspect of the disclosure is a method for synthesizing a surfactant, comprising performing a chain transfer polymerization reaction with an initiator, a monomer, and a chain transfer agent to provide a hydrophilic segment having repeating units, wherein the initiator comprises a hydrophilic group comprising less than 20 carbons and the chain transfer agent comprises a hydrophobic unit comprising at least 6 carbons, wherein the hydrophilic group and the hydrophobic unit are covalently attached to the hydrophilic segment, and wherein the ratio of the molar mass of the hydrophilic segment in kg/mol to the number of carbons in the hydrophobic unit is less than about 1.

In some embodiments, the hydrophilic segment, repeating units, hydrophilic group, and hydrophobic unit of the excipient and the ratio of the molar mass of the hydrophilic segment in kg/mol to the number of carbons in hydrophobic unit may be as otherwise described herein. In some embodiments of the method for synthesizing a surfactant, the chain transfer polymerization reaction may be a RAFT polymerization reaction (i.e., the CTA is a RAFT CTA)

The person of ordinary skill in the art will appreciate that the initiator may be any of a variety of radical polymerization initiators known in the art. In some embodiments, the initiator may be a thermal initiator, photoinitiator, or a redox initiator. In some embodiments, the initiator may be an organic peroxide such as, for example, dilauroyl peroxide, dicumoyl peroxide, benzoyl peroxide, etc., or an azo compound such as, for example, azobisisobutyronitrile, 4,4′-azobis(4-cyanovaleric acid), etc. In some embodiments, the initiator is a redox system such as, for example, benzoyl peroxide/dimethyl toluidine and ammonium persulfate/N, N,N′,N′-tetramethylethylenediamine, etc. In some embodiments, the initiator is a photoinitiator such as, for example, metal iodides, metal alkyls, and azo compounds, etc.

In one example, the initiator is an azobisnitrile comprising a carboxylic acid group, the CTA is a RAFT CTA comprising an alkyl trithiocarbonate group, and the monomer is an acrylamide.

Another aspect of the disclosure is a method for preparing a solid formulation including a biologically active compound having low water solubility, comprising forming a composition comprising a surfactant or a mixture of surfactants as otherwise described herein, and the biologically active compound, together with a solvent or mixture thereof, and removing the solvent(s) from the composition. In some embodiments, the solvent is a polar solvent or mixture of polar solvents, e.g., water, methanol, and the like. In some embodiments, the ratio of biologically active compound to the surfactant or mixture of surfactants is from about 1:99 to about 50:50, e.g., from about 1:99 to about 40:60, or from about 1:99 to about 30:70, or from about 1:99 to about 20:80, or from about 1:99 to about 15:85, or from about 1:99 to about 10:90, or the ratio is about 1:99, or about 5:95, or about 10:90, or about 15:85, or about 20:80, or about 25:75, or about 30:70, or about 35:65, or about 40:60, or about 45:55, or about 50:50, based on weight (i.e., wt. %). In some embodiments, the amount of biologically active compound and the mixture in the solvent is from about 0.1 wt. % to about 20 wt. %, e.g., from about 0.1 wt. % to about 17.5 wt. %, or from about 0.1 wt. % to about 15 wt. %, or from about 0.1 wt. % to about 12.5 wt. %, or from about 0.1 wt. % to about 10 wt. %, or from about 0.1 wt. % to about 7.5 wt. %, or from about 0.1 wt. % to about 5 wt. %, or the amount is about 0.1 wt. % of the composition, or about 0.25 wt. %, or about 0.5 wt. %, or about 0.75 wt. %, or about 1 wt. %, or about 1.25 wt. %, or about 1.5 wt. %, or about 1.75 wt. %, or about 2 wt. %, or about 2.25 wt. %, or about 2.5 wt. %, or about 2.75 wt. %, or about 3 wt. %, or about 3.25 wt. %, or about 3.5 wt. %, or about 3.75 wt. %, or about 4 wt. %.

Another aspect of the disclosure is a method for preparing a solid formulation including a biologically active compound having low water solubility, and a surfactant or a mixture of surfactants as otherwise described herein, comprising spray drying a solution comprising the biologically active compound and the surfactant or mixture, freeze drying a solution comprising the biologically active compound and the surfactant or mixture, or hot-melt extruding intermediate mixture of the biologically active compound and the surfactant or mixture. The person of ordinary skill in the art will appreciate that the procedures and conditions for such methods may be optimized, as is known in the art, to achieve a formulation with desirable properties.

Another aspect of the disclosure is a method for reconstituting a solid formulation including a biologically active compound having low water solubility and a surfactant or mixture of surfactants as otherwise described herein, comprising forming a mixture comprising the solid formulation and a polar solvent, and agitating the composition.

Another aspect of the disclosure if a method for applying a biologically active compound having low water solubility to a substrate, comprising contacting the substrate with a formulation or micellar composition including the biologically active compound and a surfactant or a mixture of surfactants as otherwise described herein.

Another aspect of the disclosure is a method for delivering a biologically active compound having low water solubility to an animal, comprising administering to the animal a formulation or micellar composition including the biologically active compound and a surfactant or a mixture of surfactants as otherwise described herein. In some embodiments, the biologically active compound is administered orally.

Yet another aspect of the disclosure is a formulation including a surfactant or a mixture of surfactants as otherwise described herein, hydroxypropyl methylcellulose acetate succinate (HPMCAS), and a biologically active compound having low water solubility. In some embodiments, the concentration of HPMCAS in the formulation is within the range of about 40 wt. % to about 95 wt. %, or about 45 wt. % to about 95 wt. %, or about 50 wt. % to about 95 wt. %, or about 55 wt. % to about 95 wt. %, or about 60 wt. % to about 95 wt. %, or about 65 wt. % to about 95 wt. %, or about 70 wt. % to about 95 wt. % of the formulation. In some embodiments, the formulation is a solid formulation. In some embodiments, the formulation may be prepared according to the methods as otherwise described herein.

### EXAMPLES

The Examples that follow are illustrative of specific embodiments of the invention, and various uses thereof. They are set forth for explanatory purposes only, and are not to be taken as limiting the invention.

### Example 1. CTA Synthesis

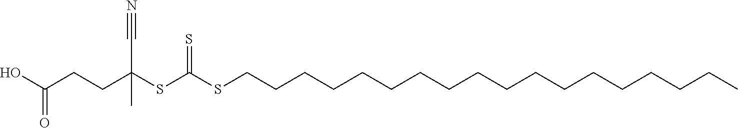 [SMILES: CCCCCCCCCCCCCCCCCCSC(=S)SC(C)(C#N)CCC(=O)O]

The synthesis of 4-cyano-4-[(octadecylsulfanylthiocarbonyl)sulfanyl]pentanoic acid (shown above) was adapted from a procedure by Moad et al. (Polymer 2005, 46, 8458-8468). 1-Octadecanethiol (21.78 g, 76 mmol) was dissolved in 200 mL of diethyl ether, and was added over 10 min to a stirred suspension of sodium hydride (60% in oil) (3.15 g, 79 mmol) in diethyl ether (250 mL) at 5° C. Evolution of hydrogen was observed as the suspension developed into a thick, white slurry. The reaction mixture was cooled to 0° C. and carbon disulfide (6.0 g, 79 mmol) was added, turning the slurry to a bright yellow color. The solids were collected by filtration and used without purification.

A suspension of sodium S-octadecyl trithiocarbonate (39.24 g, 102 mmol) in diethyl ether (400 mL) was treated by portion-wise addition of solid iodine. The reaction stirred for 1 h at room temperature. Sodium iodide and unreacted S-octadecyl trithiocarbonate were removed by filtration, redissolved in diethyl ether, treated with additional solid iodine for 1 h, and filtered again. The yellow-brown filtrates were combined, and washed with aqueous sodium thiosulfate until the organic layer was a clear, yellow color. The organic layer was dried over sodium sulfate, filtered, and evaporated to leave a bright yellow powder of bis(octadecaylsulfanylthiocarbonyl) disulfide.

A solution of bis(octadecaylsulfanylthiocarbonyl) disulfide (16.0 g, 22.1 mmol) and 4,4′-azobis(4-cyanopentanoic acid) (9.31 g, 33.2 mol) in ethyl acetate (450 mL) was heated at reflux for 20 h. After removal of volatiles in vacuo, the crude product was rinsed 5× with 400 mL DI water to remove water-soluble side products. The crude mixture was evaporated down, redissolved in minimal ethyl acetate, and purified by column chromatography on silica with a 75/25 hexanes/ethyl acetate mobile phase supplemented with 1% acetic acid (R_{f}=0.2). The fractions were combined and evaporated to a crude solid. The solid was then dissolved in ethyl acetate and extracted into an aqueous phase of NaHCO₃. After isolation of the aqueous phase, the compound was extracted back into an organic phase of ethyl acetate through acidification of the aqueous layer with 1 M HCl. The organic phase was evaporated to yield a product of 4-cyano-4-[(octadecylsulfanylthiocarbonyl)sulfanyl]pentanoic acid. Anal Calcd for C₂₅H₄₅NO₂S₃: C, 61.55; H, 9.30; N, 2.87; S, 19.72. Found: C, 61.43; H, 9.37; N, 2.96; S, 18.48. HRMS (ESI-TOF) m/z: [M+Na]⁺ Calcd for C₂₅H₄₅NO₂S₃Na 510.2510; Found 510.2511.

### Example 2. Surfactant Synthesis

The syntheses of a group of surfactants including a RAFT group linker, an alkyl hydrophobic unit, and an N-isopropylacrylamide-derived repeating unit were carried out according to similar procedures. An exemplary procedure is provided here. N-isopropylacrylamide (1.275 g, 0.01127 mol), 4-cyano-4-[(octadecylsulfanylthiocarbonyl) sulfanyl]pentanoic acid (0.500 g, 0.00102 mol), and 4,4′-azobis(4-cyanopentanoic acid) (0.0286 g, 0.000102 mol) were added to a round-bottom flask. Dioxane (25 mL) was added. The reaction mixture was sparged with nitrogen gas for 45 minutes, and then placed in an oil bath at 72° C. After at least 7 hours, the reaction was quenched by cooling to 0° C. and opening the flask to air. The resultant polymer was isolated and purified by precipitation into a 75/25 v/v mixture of hexanes/diethyl ether (2×). The polymer was dried under vacuum at 40° C. overnight to yield the final polymer (87% yield).

Surfactants having structural formula II (shown below) were prepared according to the above procedure and include a range of varying hydrophilic first end groups (R), hydrophilic segment lengths, and alkyl hydrophobic units, as described in Tables 1-2, below.

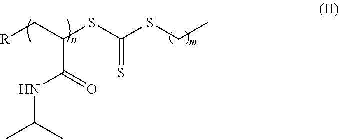

TABLE 1
R Groups of Formula II

| R Group | Structure |
|---|---|
| 1 | 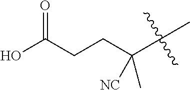 [SMILES: *C(C)(C#N)CCC(=O)O] |
| 2 | 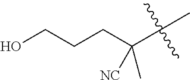 [SMILES: *C(C)(C#N)CCCO] |
| 3 | 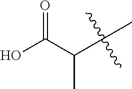 [SMILES: *C(C)C(=O)O] |
| 4 | 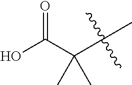 [SMILES: *C(C)(C)C(=O)O] |
| 5 | 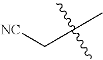 [SMILES: *CC#N] |
| 6 | 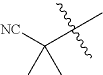 [SMILES: *C(C)(C)C#N] |
| 7 | 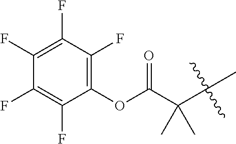 [SMILES: *C(C)(C)C(=O)Oc1c(F)c(F)c(F)c(F)c1F] |

TABLE 2
Example Structures

| Example | R Group | m |
|---|---|---|
| 1 | 1 | 11 |
| 2 | 2 | 11 |
| 3 | 3 | 11 |
| 4 | 1 | 5 |
| 5 | 1 | 17 |
| 6 | 1 | 17 |
| 7 | 4 | 11 |
| 8 | 1 | 5 |
| 9 | 1 | 11 |
| 10 | 1 | 17 |
| 11 | 5 | 11 |
| 12 | 6 | 11 |
| 13 | 7 | 11 |
| 14 | 1 | 1 |
| 15 | 1 | 1 |
| 16 | 1 | 11 |

1H NMR and 13C NMR spectroscopy experiments were performed on a Bruker Avance III HD 500 with a 5-mm Prodigy TCI cryoprobe at 500 MHz and 125 MHz, respectively. Samples were dissolved in DMSO-d6 or CDCl3 as the solvent. Size exclusion chromatography (SEC) was carried out in THF on an Agilent 1260 liquid chromatograph. The SEC housed three Phenomenex Phenogel-5 columns connected in series, and solvent ran at a flow rate of 1 mL min-1 at 25° C. The detectors include an Agilent 1260 VWD UV-Vis detector, a Wyatt Dawn DSP Heleos II multi-angle light scattering (MALS) detector, and a Wyatt Optilab T-rEX refractive-index detector. Elemental analysis was performed by Atlantic Microlab, Inc (Norcross, Ga.). High-resolution mass spectrometry was performed on a Bruker BioTOF II instrument using a mixture of poly(ethylene glycol)s as the internal calibrant. Characterization data for Examples 1-11 and Comparative Examples 1-6 are shown in Table 3.

TABLE 3
Surfactant Characterization

| | Mₙ (NMR) | Mₙ (SEC) | M_{w} (SEC) | | T_{g} | |
|---|---|---|---|---|---|---|
| Example | (kg/mol) | (kg/mol) | (kg/mol) | Ð | (° C.) | Ratio* |
| 1 | 1.3 | 2.0 | 2.0 | 1.02 | 70 | 0.11 |
| 2 | 1.3 | 2.1 | 2.2 | 1.08 | 74 | 0.11 |
| 3 | 1.5 | 2 | 2.2 | 1.1 | 75 | 0.12 |
| 4 | 2.1 | 2.6 | 2.8 | 1.08 | 95 | 0.35 |
| 5 | 4.6 | 4.8 | 5.0 | 1.05 | 98 | 0.26 |
| 6 | 6.0 | 6.0 | 6.1 | 1.02 | 96 | 0.33 |
| 7 | 1.6 | 1.9 | 2.2 | 1.10 | 80 | 0.13 |
| 8 | 3.8 | 4.6 | 4.8 | 1.02 | 101 | 0.63 |
| 9 | 4.4 | 4.5 | 4.6 | 1.03 | 101 | 0.37 |
| 10 | 8.0 | 8.1 | 8.3 | 1.02 | 111 | 0.44 |
| 11 | 1.5 | 5.7 | 6.0 | 1.06 | 72 | 0.12 |
| 12 | 1.4 | 4.7 | 5.3 | 1.14 | 70 | 0.12 |
| 13 | 1.5 | 2.7 | 2.9 | 1.08 | 79 | 0.12 |
| 14 | 1.8 | 2.3 | 2.6 | 1.13 | 98 | 0.9 |
| 15 | 3.9 | 3.9 | 4.0 | 1.02 | 102 | 1.95 |
| 16 | 22 | 21.4 | 44.2 | 1.04 | 114 | 1.83 |

*Ratio of the molar mass of the poly(N-isopropylacrylamide) hydrophilic segment in kg/mol to (m + 1).

### Example 3. Spray-Dried Formulation Preparation

Spray-dried dispersions including a phenytoin drug (shown below) were prepared at lab-scale using a Mini-spray dryer (Bend Research, Bend, Oreg.). Solid dispersions were made at 10 wt. % and 25 wt. % drug (90 and 75 wt. % polymer, respectively). At a total solids loading of 2.0 wt. %, drug and polymer were dissolved in a 90/10 v/v MeOH/H₂O solution, unless otherwise specified. Samples were sprayed at a constant inlet temperature (75° C.), N₂ (flow rate 28.6 sL min⁻¹), and solution flow rate (1.3 mL min⁻¹). Spray-dried dispersions were collected and immediately dried under vacuum overnight, and further stored at room temperature in a vacuum desiccator.

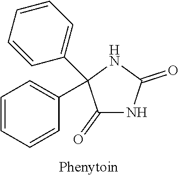 [SMILES: O=C1NC(=O)C(c2ccccc2)(c2ccccc2)N1]

Such a formulation, wherein the drug (i.e., the API) is rendered amorphous in a solid state within a mixture of the surfactant, is in a form that may be administered orally, rather than, e.g., injected intravenously.

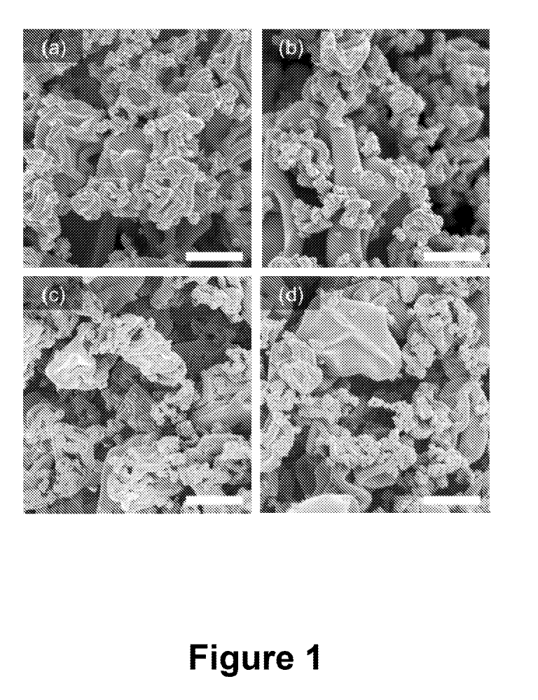

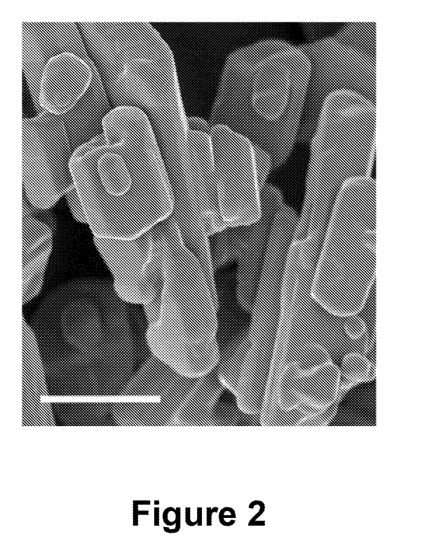

Dispersions were imaged with scanning electron microscopy (SEM) using a Hitachi S-4700 field emission gun SEM using a secondary electron detector operating at an accelerating voltage of 3.0 kV. Magnification was between 5,000× and 20,000×. Prior to imaging, Dispersions were sprinkled on to conductive carbon tape (Ted Pella Inc.) and sputter coated with 10 nm of Pt in a VCR Group IBS TM200S Ion Beam Sputterer. The SEM images, shown in FIG. 1, revealed collapsed and wrinkled particle structures ranging from 50 nm to 10 μm in size for all Dispersions. Crystalline phenytoin (See FIG. 2) was not visible in the Dispersions.

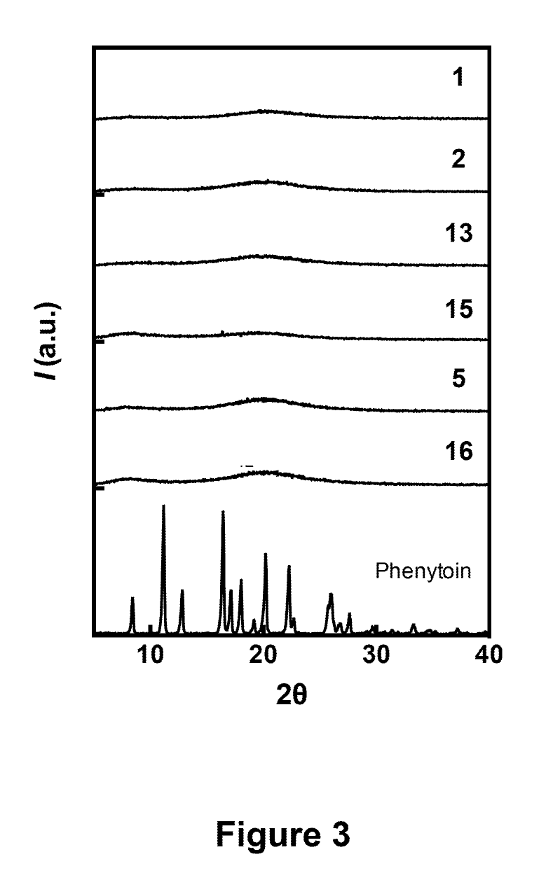

Dispersions were also examined by powder wide-angle X-ray diffraction (PXRD) using a Bruker-AXS D5005 Diffractometer. Samples were packed in a zero-background holder and analyzed using a Cu-Kα X-ray source (λ=1.54 Å, 45 kV and 40 mA). Data was collected from 5° to 40° on the 20 scale at a step size of 0.02° with a dwell time of 1 s step⁻¹. Results (See FIG. 3) showed broad, featureless spectra.

Thermogravimetric analysis (TGA) was performed on the Dispersions using a Q500 (TA Instruments, New Castle, Del.) at a heating rate of 10° C. min⁻¹ under N₂. Differential scanning calorimetry (DSC) was also performed, using a Discovery DSC (TA Instruments, New Castle, Del.) equipped with a refrigerated cooling system. A dry N₂ purge flowed through the cell at 50 mL min⁻¹, and samples were run in hermetically sealed TZero aluminum pans. Polymer samples (2-10 mg) were ramped at 10° C. min⁻¹ between −85 and 150° C. T_{g} values were taken from the second heat. Dispersion samples (2-6 mg) were ramped at 5° C. min⁻¹ from −85 and 210° C. Cold crystallization analyses and T_{g} values were taken from the first heat. Results, shown in Table 4, showed 5 wt % mass loss temperatures (T_{d,5}%) between 181 and 216° C. DSC of the solid dispersions revealed broad T_{g} values in the range of 46 to 98° C., with no evidence of phenytoin cold crystallization. Samples with molar masses above 1.8 kg mol⁻¹ showed T_{g} values above 75° C. (50° C. greater than room temperature), suggesting these solid dispersions would be suitable for storage at ambient temperatures. Nearly all T_{g} values of the ASDs were lower than the T_{g} values of the polymers alone, which can be attributed to the plasticizing effect of dispersed drug in the polymeric matrix.

TABLE 4
Thermal Characterization of Dispersions

| | T_{d,5%} (° C.)^{a} | T_{g} (° C.)^{b} |
|---|---|---|
| Example | (TGA) | (DSC) |
| 1 | 207 | 78 |
| 2 | 201 | 66 |
| 3 | 202 | 70 |
| 4 | 198 | 84 |
| 5 | 204 | 96 |
| 6 | 216 | 94 |
| 7 | 198 | 70 |
| 8 | 203 | 91 |
| 9 | 202 | 97 |
| 10 | 202 | 97 |
| 11 | 181 | 49 |
| 12 | 209 | 65 |
| 13 | 213 | 46 |
| 14 | 210 | 89 |
| 15 | 209 | 93 |
| 16 | 201 | 98 |

^{a}Temperature at 5 wt % mass loss by thermogravimentric analysis in nitrogen.
^{b}Glass transition temperatures (T_{g}s) of the solid dispersions as determined by DSC in the second heating cycle at a heating rate of 10° C. min⁻¹.

### Example 4. Dried Formulation Dissolution Test

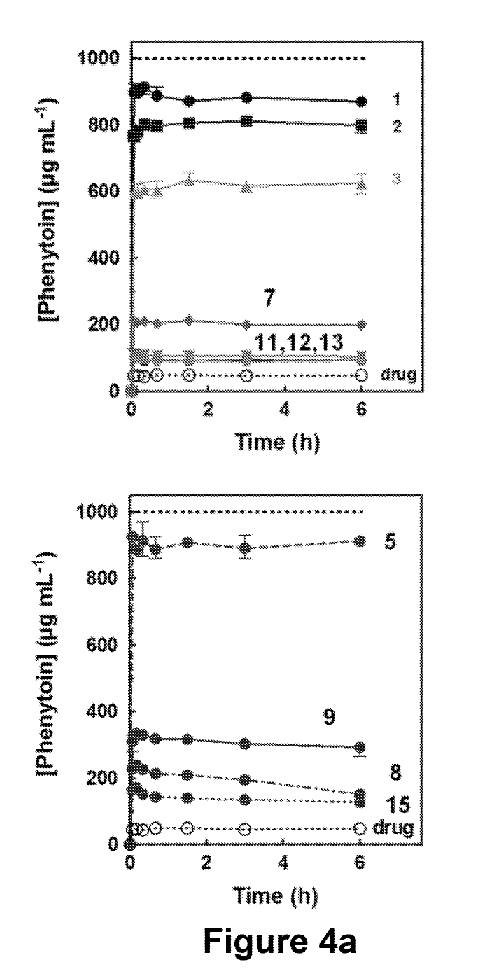

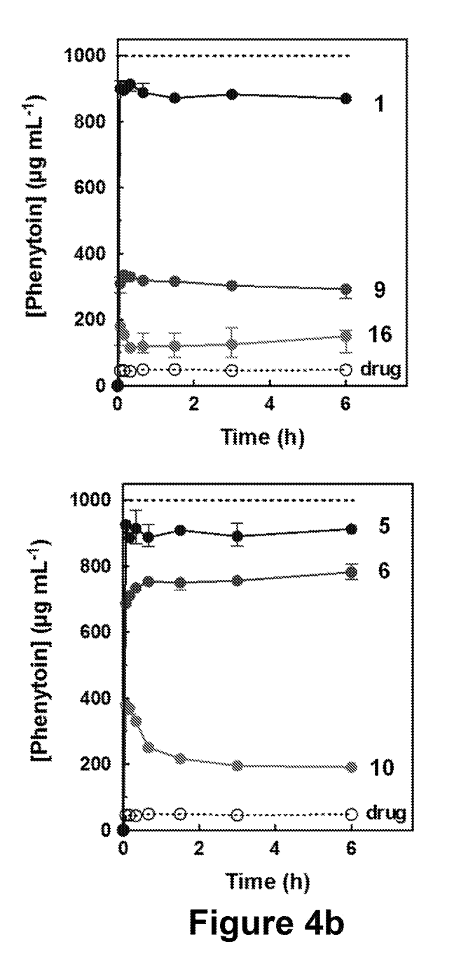

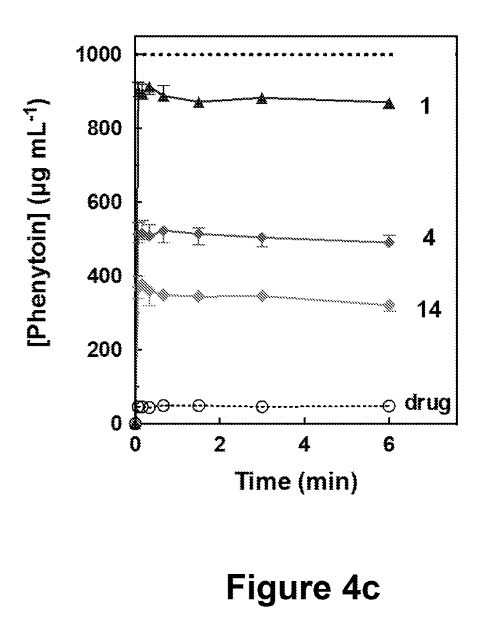

Dissolution tests were performed in phosphate buffered saline solution (PBS, 82 mM sodium chloride, 20 mM sodium phosphate dibasic, 47 mM potassium phosphate monobasic) with 0.5 wt % simulated intestinal fluid (SIF) powder (FaSSIF, Biorelevant) at 37° C. In a 1.5-mL microcentrifuge tube, an appropriate amount of spray-dried dispersion and PBS was added to target a total drug concentration of 1000 μg mL-1. Samples were vortexed to suspend all solids for 1 min and immediately set in an isothermal sample holder held at 37° C. (t=0). At set time points (4, 10, 20, 40, 90, 180, 360 min), the samples were centrifuged at 13,000 g for 1 min, a 50-μL aliquot was removed from the supernatant, and the aliquot was diluted with 300 μL of HPLC-grade methanol. The samples were then vortexed for 30 sec to resuspend the dispersion and returned to the isothermal sample holder. The drug concentration was determined by high performance liquid chromatography in 60/40 (v/v) H₂O/MeCN. Samples were analyzed on an Agilent 1260 liquid chromatograph with a multi-wavelength UV-Vis detector, 1260 MWD, at 225 nm. Separation occurred on an Agilent Poroshell 120 EC-C18 column with 120 Å pores. Results are shown in Table 5, below, and representative dissolution profiles are provided in FIG. 4.

TABLE 5
Formulation Dissolution Data

| | cₘₐₓ | c_{6 h} | AUC_{6 h} | | |
|---|---|---|---|---|---|
| Example | (μg mL⁻¹)^{c} | (μg mL⁻¹)^{c} | (h⁻¹ μg mL⁻¹)^{c} | EF (EF %)^{d} | |
| 1 | 910 | 870 | 5250 | 18.6 | (87%) |
| 2 | 810 | 800 | 4800 | 17.0 | (80%) |
| 3 | 630 | 620 | 3700 | 13.1 | (62%) |
| 4 | 380 | 320 | 3020 | 10.7 | (50%) |
| 5 | 926 | 913 | 5360 | 19.1 | (90%) |
| 6 | 780 | 780 | 4520 | 16.0 | (75%) |
| 7 | 210 | 200 | 1210 | 4.3 | (20%) |
| 8 | 240 | 150 | 1130 | 4.1 | (19%) |
| 9 | 335 | 293 | 1830 | 6.5 | (31%) |
| 10 | 380 | 190 | 1290 | 4.6 | (22%) |
| 11 | 110 | 100 | 620 | 2.2 | (10%) |
| 12 | 110 | 100 | 560 | 1.9 | (9%) |
| 13 | 110 | 100 | 540 | 1.9 | (9%) |
| 14 | 520 | 490 | 2040 | 7.2 | (34%) |
| 15 | 170 | 130 | 820 | 2.9 | (14%) |
| 16 | 180 | 150 | 510 | 2.8 | (13%) |

^{c}Dissolution performance metrics, where cₘₐₓ is the maximum phenytoin concentration achieved over the 6-h study, c_{6 h} is the final concentration at the 6 h time point, AUC_{6 h} is the area under the concentration-time curve over the 6 h study.
^{d}The enhancement factor (EF) is defined as EF = AUC_{6 h} (solid dispersion)/AUC₆ₕ(phenytoin).

Based on these studies, if all loaded drug was released completely and sustained at 1000 μg mL⁻¹, the maximum EF possible is 21.3. The parenthetical percentage (%) is the percentage of the max possible enhancement factor (21.3) based on drug loading.

These results demonstrate a rapid, or “burst” release of the drug in conditions mimicking intestinal fluid, and complete maintenance of an enhanced solubility (i.e., supersaturation). Without being bound by a particular theory, these features are thought to be due to the smaller size of the micellar aggregates comprising the surfactants and the drug, and may be due to interaction of the drug with the corona of the micellar aggregates, rather than solely with the hydrophobic core.

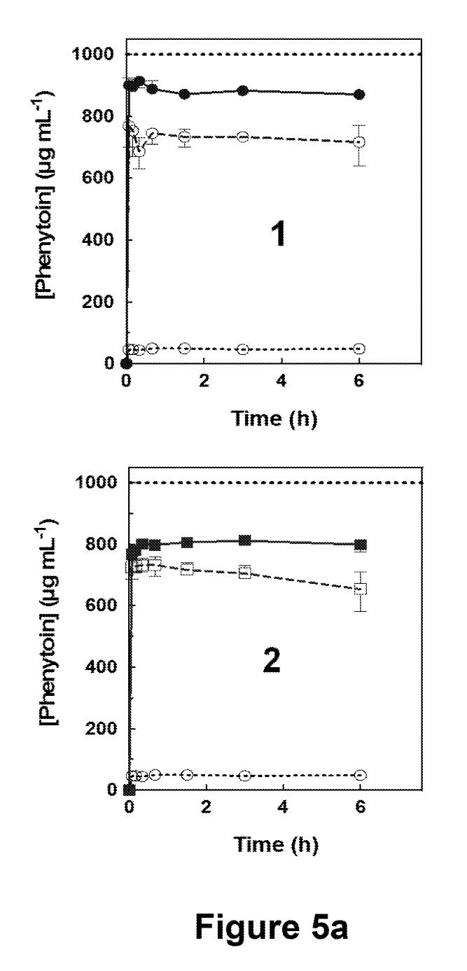

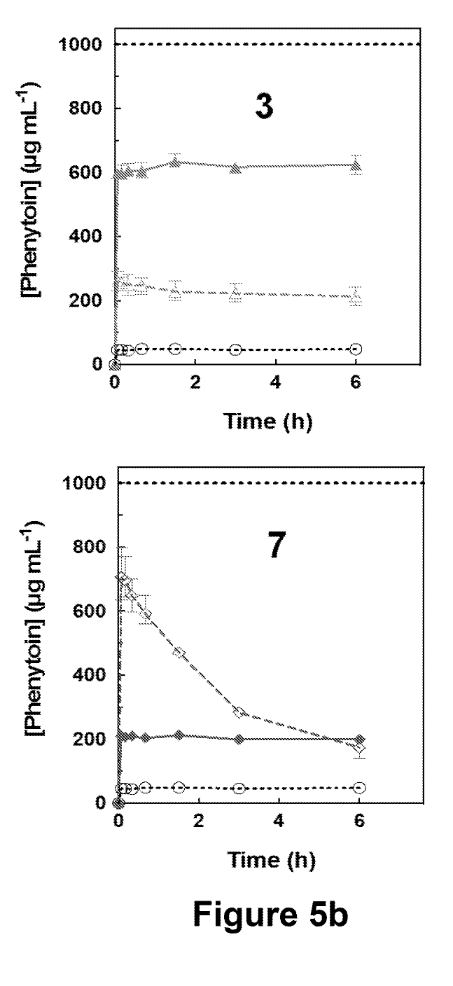

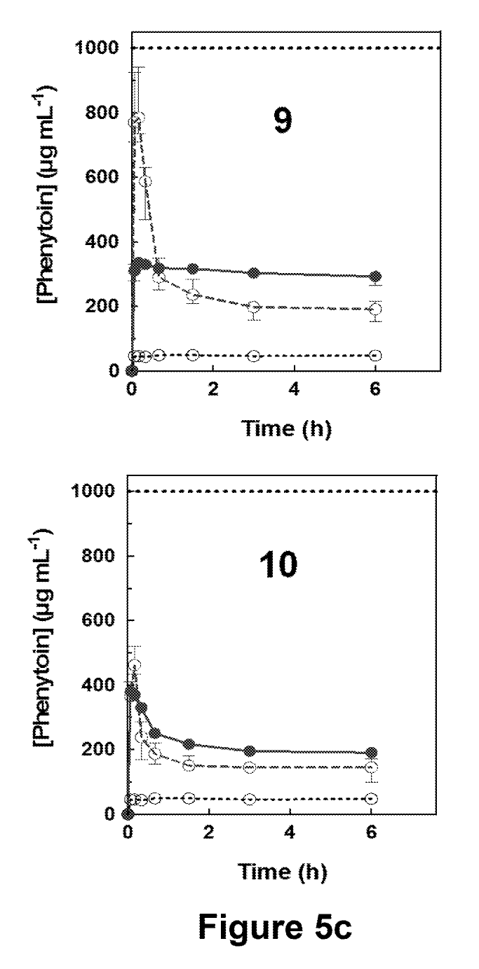

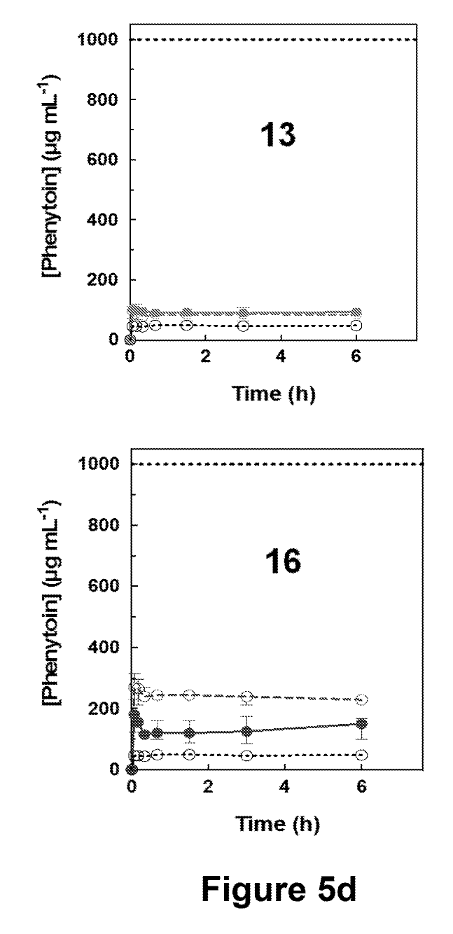

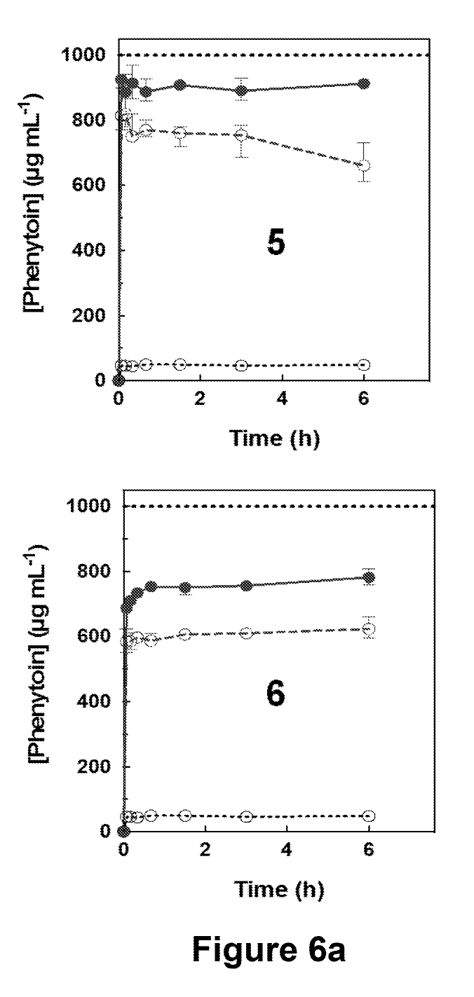

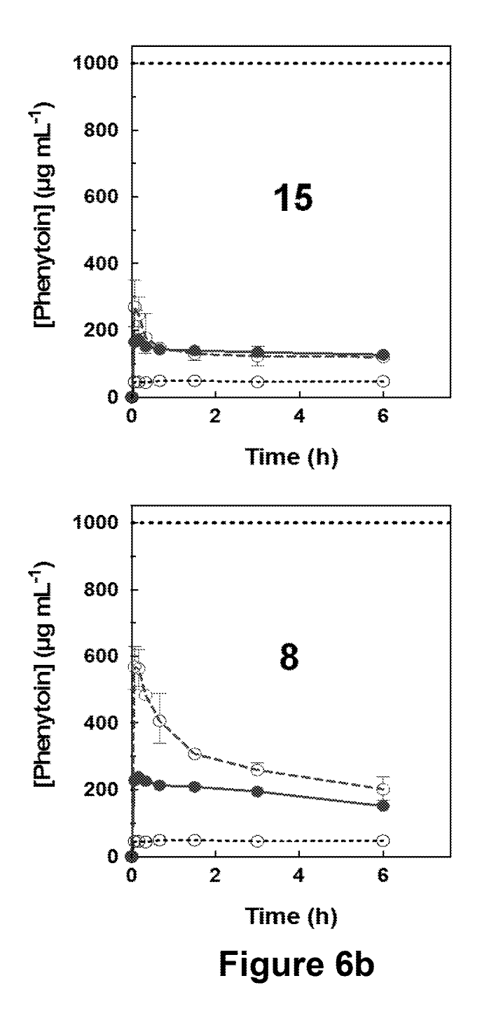

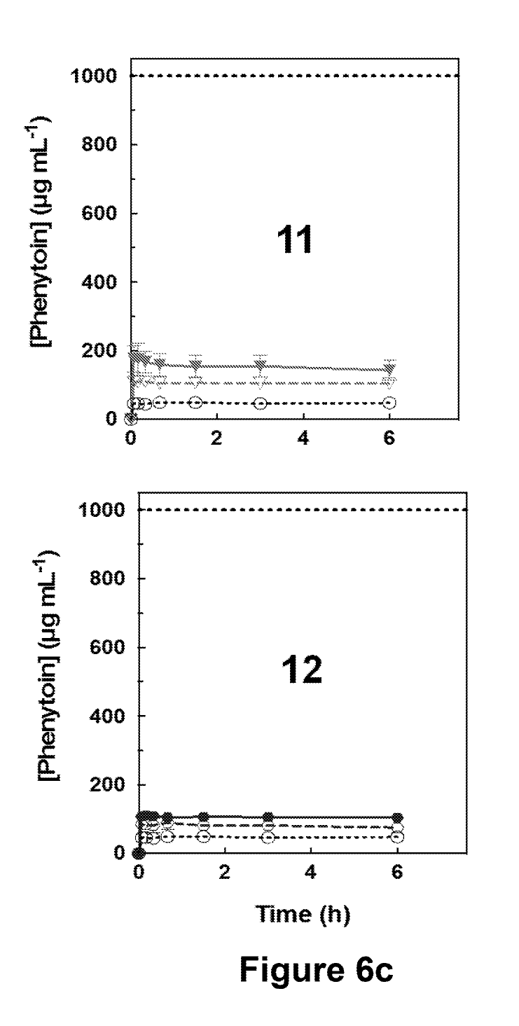

The ability of predissolved polymer to maintain a supersaturation of drug was tested by adding a concentrated stock solution of phenytoin in organic solvent (methanol) to a microcentrifuge tube with polymer predissolved in PBS (w/SIF powder). To match dissolution concentration conditions from solid dispersions with 10 wt % drug, polymers were added at a loading of 9 mg mL⁻¹ (9,000 pg mL⁻¹) and drug was added at a loading of 1 mg mL⁻¹ (1,000 pg mL⁻¹). Polymer (˜12-13 mg) was added to a 1.5-mL microcentrifuge tube, and an appropriate amount of PBS w/0.5 wt % SIF was added to reach a 9 mg mL⁻¹ solution of polymer. The tube was vortexed on a Scientific Industries Vortex-Genie 2 on a setting of 10 for 1 min and then set in an isothermal sample holder at 37° C. for 30 min. Separately, a stock solution of phenytoin was prepared in methanol at 0.02 mg μL⁻¹. After the 30 min equilibration, the solution of polymer was revortexed for 1 min, and then the amount of phenytoin stock solution necessary to reach 1 mg mL⁻¹ was added. The solution was vortexed for 1 min and returned to the isothermal sample holder. Time points were taken as described earlier for dissolution testing (4, 10, 20, 40, 90, 180, and 360 min). Concentrations were determined as described above. Results are provided in FIGS. 5-6.

### Example 5. Additional Drug Investigations

Spray-dried formulations including either oxaprozin or 5-methyl-5-phenylhydantoin (5-m-5-p) (shown below) and a surfactant prepared according to Example 2, wherein R=1 and m=11, were prepared according to the procedure of Example 3 (See Table 6).

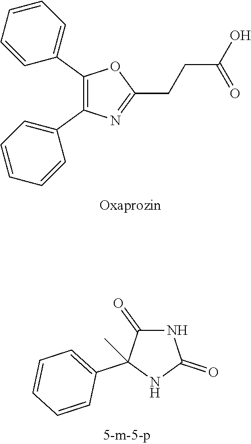 [SMILES: O=C(O)CCc1nc(-c2ccccc2)c(-c2ccccc2)o1 ; CC1(c2ccccc2)NC(=O)NC1=O]

TABLE 6
Dispersion Preparations

| | M_{w} (SEC) | | |
|---|---|---|---|
| Dispersion | (kg/mol) | Ratio* | Drug |
| A | 2 | 0.17 | Oxaprozin |
| B | 6 | 0.5 | Oxaprozin |
| C | 12 | 1.0 | Oxaprozin |
| D | 15 | 1.25 | Oxaprozin |
| E | 21 | 1.75 | Oxaprozin |
| F | 2 | 0.17 | 5-m-5-p |
| G | 6 | 0.5 | 5-m-5-p |
| H | 12 | 1.0 | 5-m-5-p |
| I | 15 | 1.25 | 5-m-5-p |
| J | 21 | 1.75 | 5-m-5-p |

*Ratio of the molecular weight of the poly(N-isopropylacrylamide) hydrophilic segment in kDa/mol to (m + 1).

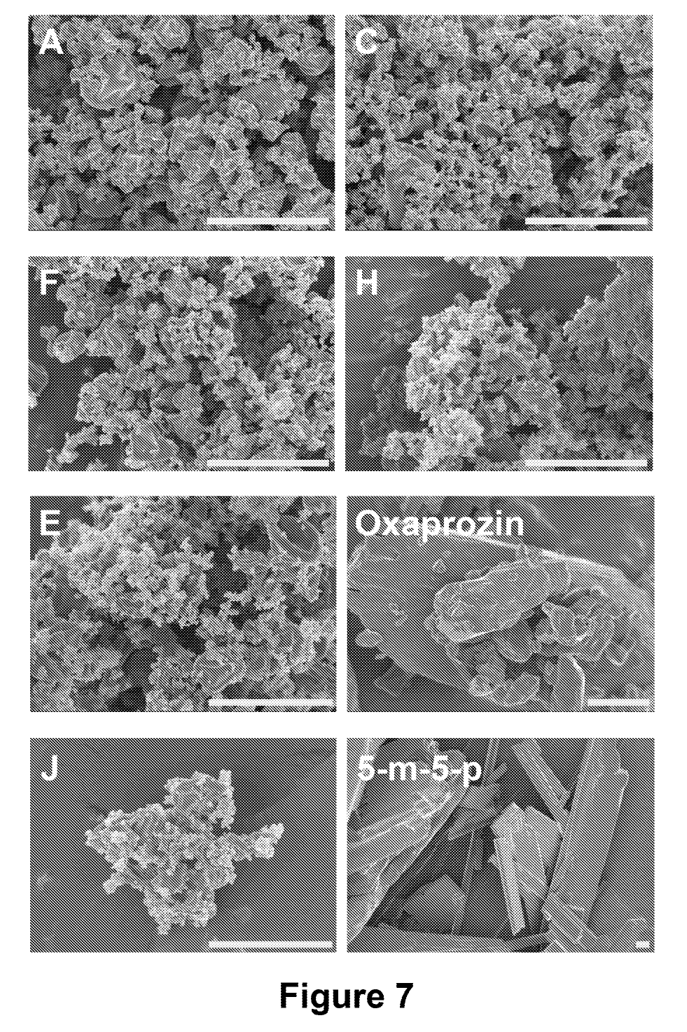

Dispersions A-J were imaged with scanning electron microscopy (SEM) using a Hitachi S4700 with an accelerating voltage of 3 kV and at magnifications of ×10 k and ×20 k. Each sample was sputter coated with 200 Å of platinum. The images, shown in FIG. 7, reveal a collapsed-sphere morphology for each dispersion

Thermogravimetric analysis (TGA) of Dispersions A-J was performed using a Q500 instrument from TA Instruments. The decomposition temperature (T_{d}) at 5% was recorded for each dispersion and T_{d,1%} was recorded for the pure drugs. T_{d,5%} was recorded for the dispersions due to the hydrophilic properties of the surfactants, which required removal of surface moisture before the materials began to decompose. Differential Scanning Calorimetry (DSC) experiments were performed on Dispersions A-J and pure oxaprozin and 5-methyl-5-phenylhydantoin using a Discovery Series instrument from TA Instruments. Each dispersion was heated twice to T_{d,5%}. The pure compounds were heated twice to T_{d,1%}. The heating rate in all experiments was 10° C./min. The TGA results, provided in Table 6, showed a similar temperature of degradation for dispersions including either drug. The DSC results, also provided in Table 7, showed an increase in T_{g} with an increase in surfactant M_{w} for dispersions including either drug. These values approached the T_{g} value of pure surfactants (135.76° C.) as the ratio of the molecular weight of the poly(N-isopropylacrylamide) hydrophilic segment in kDa/mol to (m+1) increased. Generally, the dispersions including oxaprozin yielded lower T_{g} values. Without being bound by theory, the lower T_{g} values might be attributable to the larger size of oxaprozin, which would further disrupt surfactant packing.

TABLE 7
TGA Results

| | T_{d,5%} | First Heat T_{g} | Second Heat T_{g} |
|---|---|---|---|
| Dispersion | (° C.) | (° C.) | (° C.) |
| A | 249 | 59 | 56 |
| B | 230 | 80 | 86 |
| C | 237 | 82 | 87 |
| D | 245 | 83 | 92 |
| E | 240 | 101 | 93 |
| F | 210 | 74 | 59 |
| G | 225 | 84 | 91 |
| H | 228 | 104 | 97 |
| I | 231 | 106 | 96 |
| J | 232 | 111 | 101 |
| Oxaprozin | 206* | 21 | — |
| 5-methyl-5- | 188* | 134 | — |
| phenylhydantoin | | | |

*T_{d,1%}

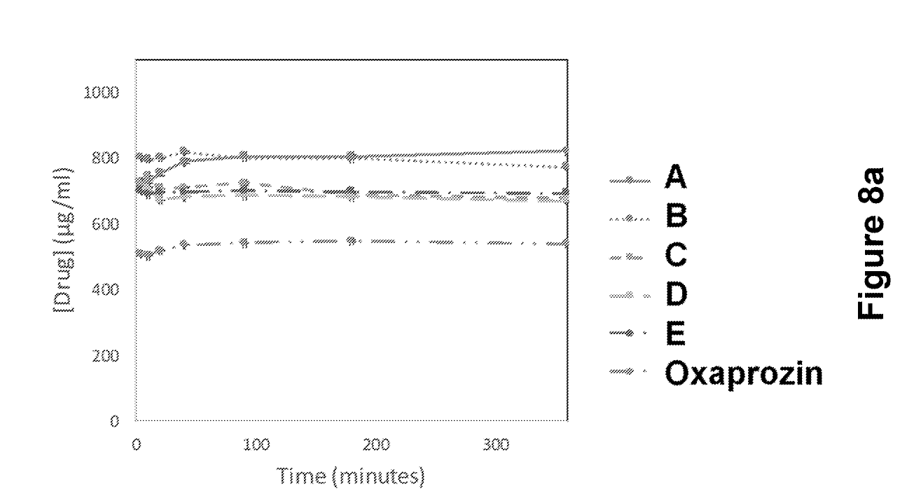

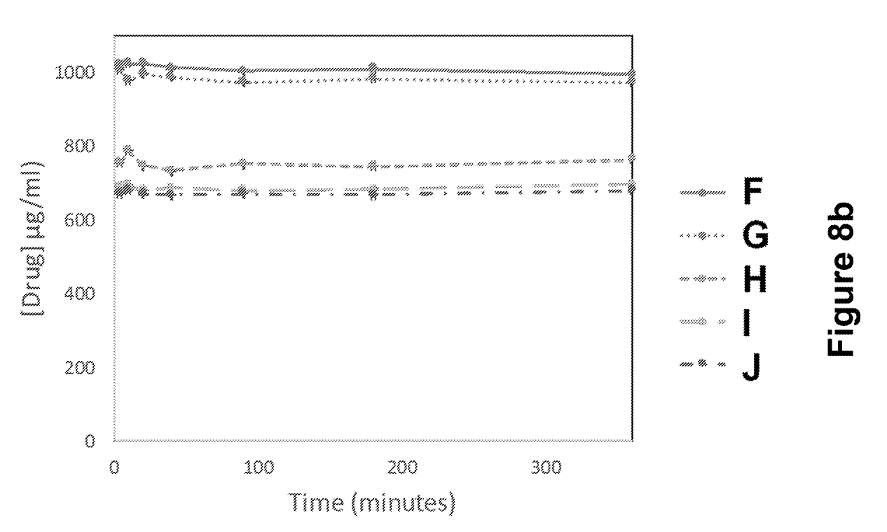

Dissolution tests of Dispersions A-J were carried out according to the procedure of Example 4. The results, shown in FIG. 8, demonstrate an enhancement in dispersions including surfactants with a molar mass of less than 10 kg/mol. Increasing performance was correlated to lower molar mass.

### Example 6. HPMCAS-Containing Formulation Investigations

Formulations including hydroxypropyl methylcellulose acetate succinate (HPMCAS) and surfactants synthesized according to Example 2 (See Table 8) were prepared.

TABLE 8
Example Structures

| Example | R | M | Ratio* |
|---|---|---|---|
| 17 | 1 | 2 | 3.5 |
| 18 | 1 | 11 | 0.58 |
| 19 | 1 | 11 | 2.5 |
| 20 | 1 | 11 | 8.2 |

*Ratio of the molar mass of the poly(N-isopropylacrylamide) hydrophilic segment in kg/mol to (m + 1).

Spray-dried dispersions including 10 wt. % phenytoin, 0-90 wt. % HPMCAS, and 90-0 wt. % surfactant (See Table 9) were prepared by dissolving the phenytoin, HPMCAS, and surfactant in THF at an overall concentration of 2 wt. %. The solution was then fed into a mini spray dryer (Bend Research, Bend, Oreg.) with the following operating conditions: solution feed rate=0.65 mL/min, nitrogen gas flow rate=12.8 standard liter per minute, inlet temperature=72° C. and outlet temperature≈25° C. The dispersions were collected on filter paper and dried under vacuum at room temperature for at least 24 hours to remove residual solvent. Dispersions were stored in a desiccator under vacuum at room temperature until used.

TABLE 9
Dispersion Compositions

| | Surfactant | HPMCAS | Surfactant |
|---|---|---|---|
| Dispersion | (Example) | (wt. %) | (wt. %) |
| A | 17 | 70 | 20 |
| B | 18 | 70 | 20 |
| C | 19 | 70 | 20 |
| D | 20 | 70 | 20 |
| E | 17 | 80 | 10 |
| F | 18 | 80 | 10 |
| G | 19 | 80 | 10 |
| H | 20 | 80 | 10 |
| I | 17 | 0 | 90 |
| J | 18 | 0 | 90 |
| K | 19 | 0 | 90 |
| L | 20 | 0 | 90 |
| M | 19 | 90 | 0 |
| N | 19 | 60 | 30 |
| O | 19 | 45 | 45 |
| P | 19 | 30 | 60 |
| Q | 19 | 10 | 80 |

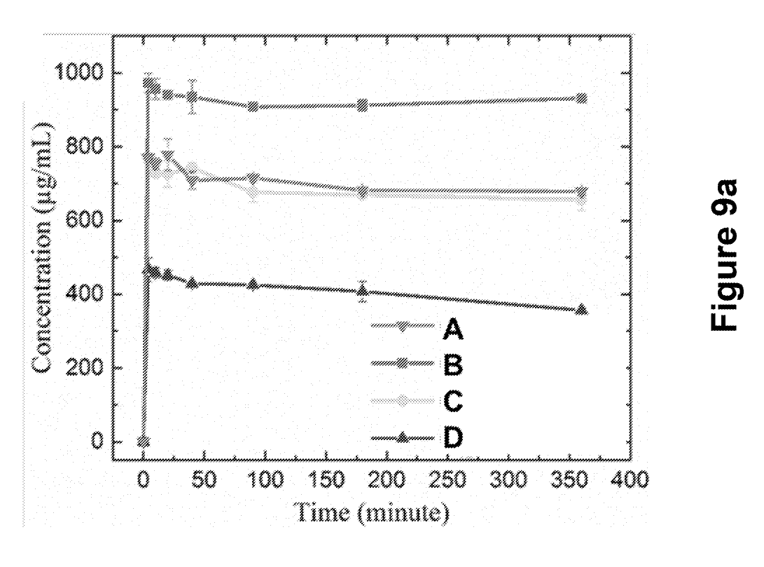

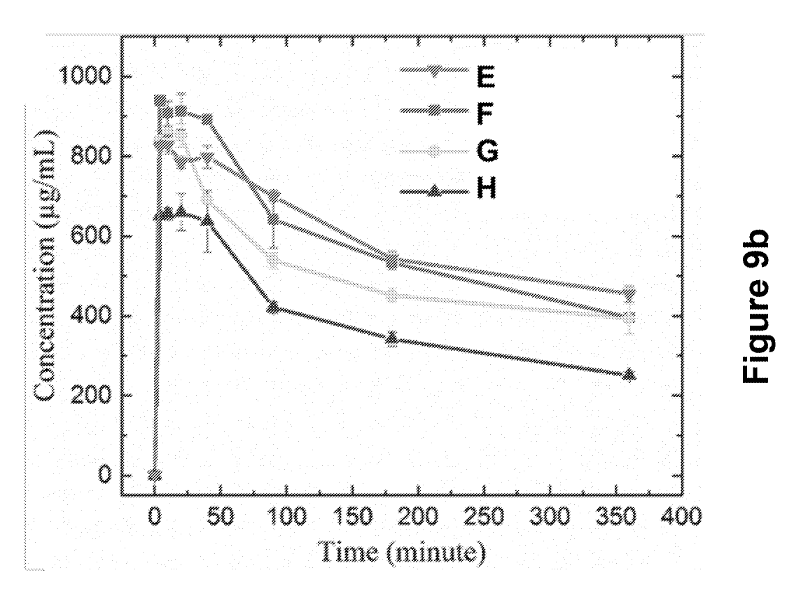

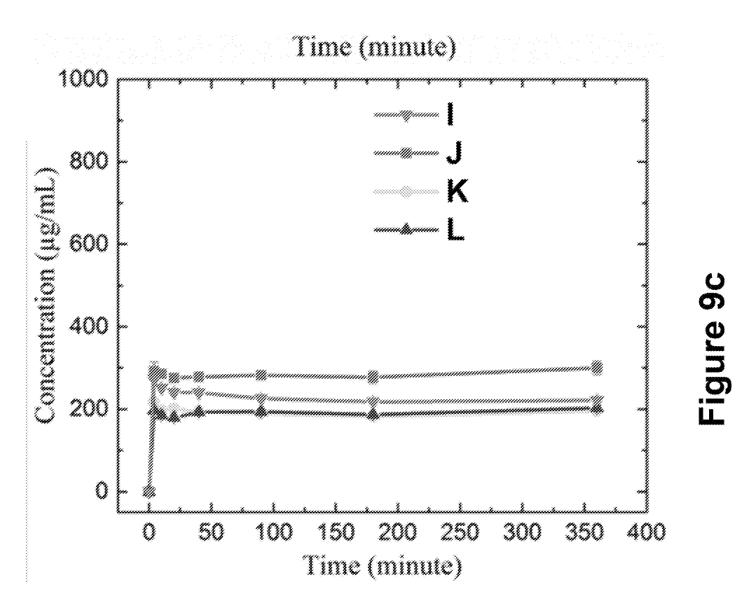

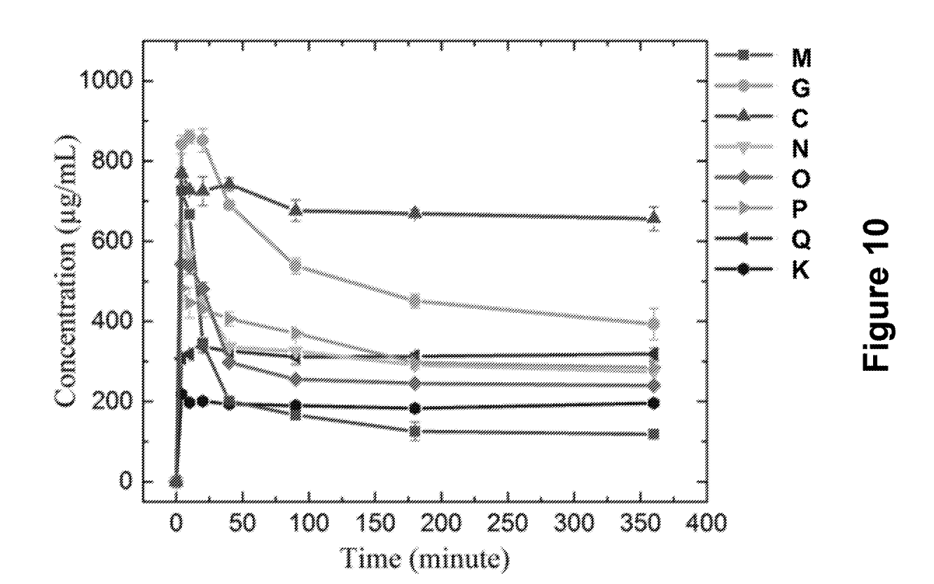

In vitro dissolution tests were carried out under non-sink conditions, over a six-hour experimental period at either 22° C. or 37° C. (i.e., below or above the cloud point of the surfactant, respectively). Dispersions were weighed into 2.0 mL plastic conical microcentrifuge tubes in duplicate. Appropriate amounts of PBS (pH=6.5) solution with 0.5 wt. % fasted simulated intestinal fluid powder (FaSSIF) were pipetted into the microcentrifuge tubes (time=0 min) to achieve a total phenytoin concentration of 1000 pg/mL if all materials were fully dissolved in the solution (e.g., 18.0 mg of Dispersion A comprising 1.8 mg of phenytoin, 12.6 mg of HPMCAS and 3.6 mg of surfactant was dissolved in 1.8 mL of PBS/FaSSIF solution). Microcentrifuge tubes were vortexed for 1 min and incubated in a VWR Analog dry heating block at either 22° C. or 37° C. At each time point (t=4, 10, 20, 40, 90, 180 and 360 min), the tubes were centrifuged at 13000 rpm for 1 min and a 50 μL aliquot from the supernatant was extracted and diluted with 250 μL methanol. The microcentrifuge tubes were then vortexed for 30 s and incubated in the heating block at either 22° C. or 37° C. until the next time point. The phenytoin concentration in the supernatant was determined by reverse phase high-performance liquid chromatography (HPLC) using a 1260 Infinity Quaternary Liquid Chromatograph System outfitted with a Poroshell 120 EC-C18 column and an Infinity Multiple Wavelength UV-Vis Detector. The mobile phase consisted of 60/40 (v/v) water/acetonitrile and the elution time of phenytoin was approximately 1.4 min. A calibration curve obtained with a wavelength of 240 nm was used to determine the phenytoin concentration. To measure the equilibrium concentration of crystalline phenytoin, an excess amount of the drug was dispersed in the PBS/FaSSIF solution and allowed to equilibrate with stirring for 48 hours at either 22° C. or 37° C. The solution was then centrifuged at 13000 rpm for 1 minute and the supernatant was extracted to determine its concentration by HPLC with the procedure described above. Results, shown in FIGS. 9 and 10, suggest that the inclusion of a carrier such as HPMCAS with a surfactant formulation as described herein enhances the solubility without limiting the release rate of a compound having low solubility in polar media.

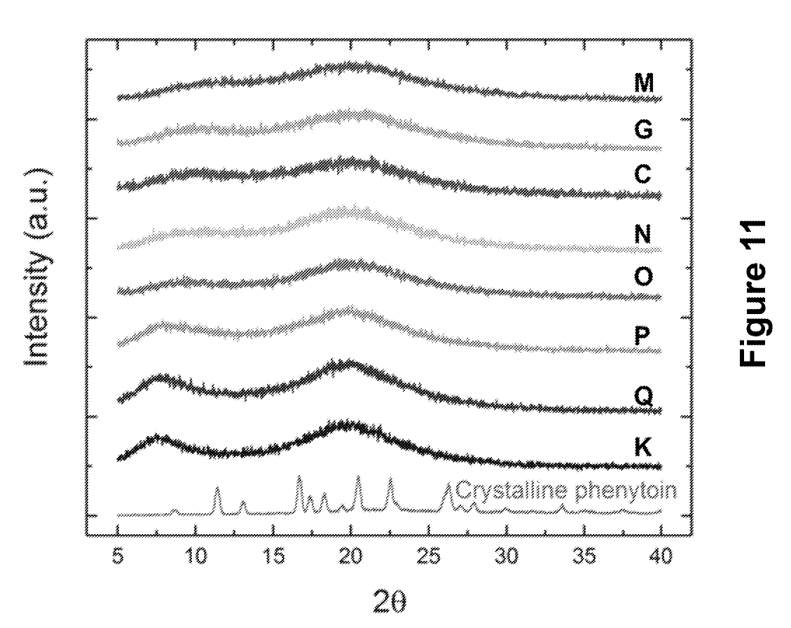

Dispersions M, G, C, N, O, P, Q, and K were subjected to wide angle X-ray scattering (WAXS) and compared to a spectrum of crystalline phenytoin. WAXS experiments were performed on a Bruker AXS (Siemens) D5005 Diffractometer with a 2.2 kW sealed Cu source (λ=1.54 Å). The instrument was calibrated with a corundum standard. Approximately 50 mg of the dispersions were loaded on a standard glass holder and inserted into the sample chamber. All experiments were performed at a voltage of 45 kV and a current of 40 mA with a 20 angle range from 5° C. to 40° (0.02° C. increments). As shown in FIG. 11, the sharp Bragg peaks that were observed for crystalline phenytoin were not observed for any of the dispersions, demonstrating a lack of crystalline phenytoin in each dispersion.

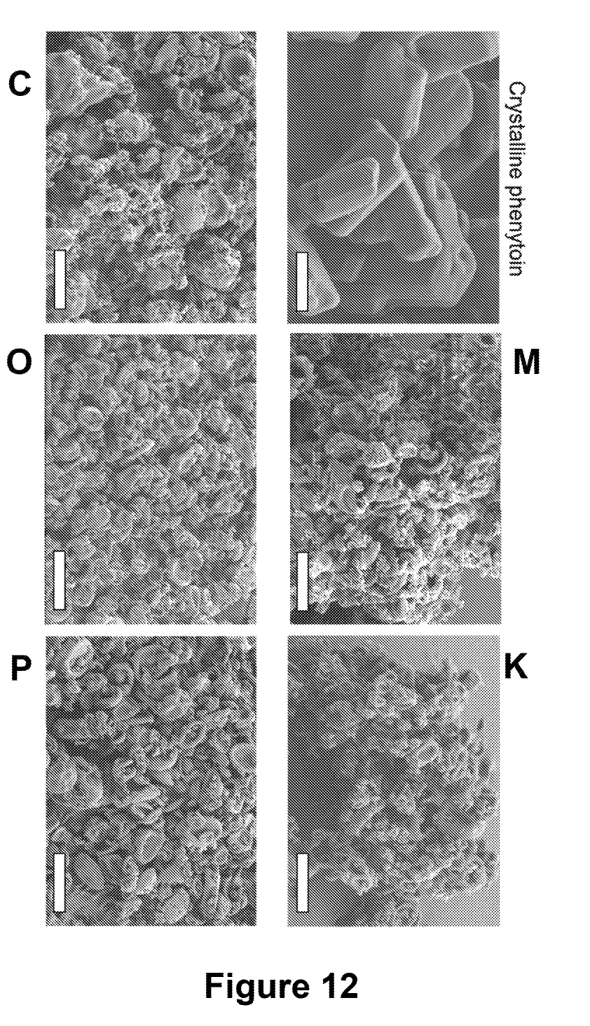

Dispersions M, K, C, O, and P were imaged with scanning electron microscopy (SEM) and compared to an SEM image of crystalline phenytoin. The representative images (See FIG. 12) demonstrate a lack of crystalline phenytoin in each dispersion.

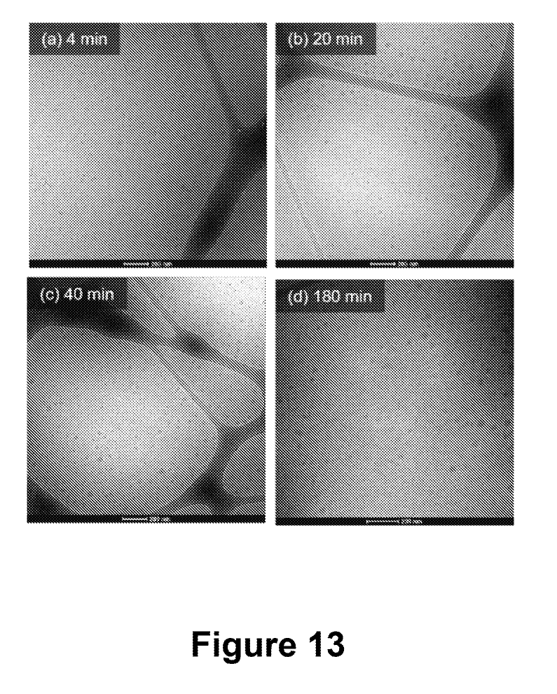

Dispersion B was imaged with cryo-transmission electron microscopy (cryo-TEM). The representative images (See FIG. 13) show micellar nanostructures.

### Example 7. Two-Surfactant-Containing Formulation Investigations

Formulations including surfactants synthesized in a manner similar to that of Example 2 (see Tables 10-11) were prepared.

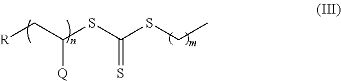

TABLE 10
Q Groups of Formula III

| Q Group | Structure |
|---|---|
| 1 | 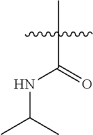 [SMILES: *C(=O)NC(C)C] |
| 2 | 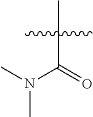 [SMILES: *C(=O)N(C)C] |
| 3 | 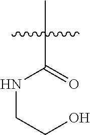 [SMILES: *C(=O)NCCO] |

TABLE 11
Example Structures

| Example | R | M | Q | Ratio* |
|---|---|---|---|---|
| 21 | 1 | 11 | 1 | 0.08 |
| 22 | 1 | 11 | 2 | 0.11 |
| 23 | 1 | 11 | 3 | 0.13 |

*Ratio of the molar mass of the hydrophilic repeating segment in kg/mol to (m + 1).

Spray-dried dispersions including 10 wt. % phenytoin, 0-90 wt. % of a first surfactant, and 90-0 wt. % of a second surfactant (see Table 12) were prepared by dissolving phenytoin and the two surfactants in 90/10 v/v methanol/water at an overall concentration of 2 wt. %. The solution was then fed into a mini spray dryer (Bend Research, Bend, Oreg.) with the following operating conditions: solution feed rate=1.3 mL/min, nitrogen gas flow rate=26.8 standard liter per minute, inlet temperature=75° C., and outlet temperature≈25° C. The dispersions were collected on filter paper and dried under vacuum at room temperature for at least 24 hours to remove residual solvent. Dispersions were stored in a desiccator under vacuum at room temperature until used.

TABLE 12
Dispersion Compositions

| | Surfactant 1 | Surfactant 1 | Surfactant 2 | Surfactant 2 |
|---|---|---|---|---|
| Dispersion | (Example) | (wt. %) | (Example) | (wt %) |
| A | 21 | 90 | 22 | 0 |
| B | 21 | 72 | 22 | 18 |
| C | 21 | 63 | 22 | 27 |
| D | 21 | 54 | 22 | 36 |
| E | 21 | 36 | 22 | 54 |
| F | 21 | 18 | 22 | 72 |
| G | 21 | 0 | 22 | 90 |
| H | 21 | 90 | 23 | 0 |
| I | 21 | 72 | 23 | 18 |
| J | 21 | 63 | 23 | 27 |
| K | 21 | 54 | 23 | 36 |
| L | 21 | 36 | 23 | 54 |
| M | 21 | 18 | 23 | 72 |
| N | 21 | 0 | 23 | 90 |

In vitro dissolution tests were carried out under non-sink conditions, over a six-hour experimental period at 37° C. Dispersions were weighed into 2.0 mL plastic conical microcentrifuge tubes in duplicate. Appropriate amounts of PBS (pH=6.5) solution with 0.5 wt. % fasted simulated intestinal fluid powder (FaSSIF) were pipetted into the microcentrifuge tubes (time=0 min) to achieve a total phenytoin concentration of 1000 pg/mL if all materials were fully dissolved in the solution. Microcentrifuge tubes were vortexed for 1 min and incubated in a VWR Analog dry heating block at 37° C. At each time point (t=4, 10, 20, 40, 90, 180 and 360 min), the tubes were centrifuged at 13000 rpm for 1 min and a 50 μL aliquot from the supernatant was extracted and diluted with 300 μL methanol. The microcentrifuge tubes were then vortexed for 30 s and incubated in the heating block at 37° C. until the next time point. The phenytoin concentration in the supernatant was determined by reverse phase high-performance liquid chromatography (HPLC) using a 1260 Infinity Quaternary Liquid Chromatograph System outfitted with a Poroshell 120 EC-C18 column and an Infinity Multiple Wavelength UV-Vis Detector. The mobile phase consisted of 60/40 (v/v) water/acetonitrle and the elution time of phenytoin was approximately 1.4 min. A calibration curve obtained was used to determine the phenytoin concentration. To analyze the dissolution of crystalline phenytoin, an excess amount of the drug was dispersed in the PBS/FaSSIF solution and time points were taken as described above for the solid dispersions. Dissolution results, shown in FIGS. 14 and 15, and the respective enhancement factors, displayed in Table 13, suggest that the inclusion of two surfactants within a single formulation as described herein enhances the solubility without limiting the release rate of a compound having low solubility in polar media.

TABLE 13
Formulation Dissolution Data

| Dispersion | EF^{a} |
|---|---|
| A | 20 |
| B | 20 |
| C | 18 |
| D | 17 |
| E | 11 |
| F | 3 |
| G | 21 |
| H | 19 |
| I | 18 |
| J | 17 |
| K | 11 |
| L | 9 |

^{a}The enhancement factor (EF) is defined as EF = AUC_{6 h}(solid dispersion)/AUC_{6 h}(phenytoin). Based on these studies, if all loaded drug was released completely and sustained at 1000 μg mL⁻¹, the maximum EF possible is 21.3

It is understood that the examples and embodiments described herein are for illustrative purposes only and that various modifications or changes in light thereof will be suggested to persons skilled in the art and are to be incorporated within the spirit and purview of this application and scope of the appended claims.

1. A surfactant consisting of a hydrophilic segment having repeating units and a first end and a second end;
the first end having a hydrophilic first end group; and
the second end having a second end group; wherein
the first end group comprises from 2 to 20 carbons;
the second end group comprises a linker and a hydrophobic unit, wherein the hydrophobic unit comprises from 6 to 40 carbons; and
the ratio of the molar mass of the hydrophilic segment in kg/mol to the number of carbons in the second end group is from about 0.05 to about 1.

2. The surfactant of claim 1, wherein the linker is a chain transfer agent (CTA) group or a reversible addition-fragmentation chain-transfer polymerization (RAFT) group.

3. (canceled)

4. The surfactant according to claim 1, wherein the ratio of the molar mass of the hydrophilic segment in kg/mol to the number of carbons in the second end group is from about 0.05 to 0.36.

5. The surfactant according to claim 1, wherein the molar mass of the hydrophilic segment is less than about 20 kg/mol.

6. The surfactant according to claim 1, wherein the repeating units are the same.

7. The surfactant of claim 6, wherein the unit of the hydrophilic segment is derived from an N-isopropylacrylamide monomer, an N,N-dimethylacrylamide monomer, or an N-hydroxyethylacrylamide monomer.

8. The surfactant according to claim 1, wherein the hydrophobic unit of the second end group is alkyl.

9. The surfactant according to claim 1, wherein the first end group comprises an alcohol or carboxylic acid.

10. A surfactant having the formula 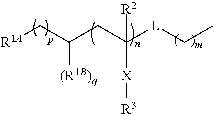
wherein:
n is from 1 to 1000;
m is from 1 to 39;
p is 0, 1, 2, or 3;
q is 0, 1, or 2;
R^{1A} is selected from C₁-C₅fluoroalkyl, C₁-C₅ hydroxyalkyl, (C₁-C₅ alkoxy)C₁-C₃alkyl, —C(O)R^{1C}, —C(S)R^{1C}, —S(O)₁₋₂R^{1C}, —C(O)OR^{1C}, —C(O)NR^{1D}R^{1C}, —C(O)SR^{1C}, —C(S)OR^{1C}, —C(S)NR^{1D}R^{1C}, —C(S)SR^{1C}, —C(NR^{1D})NR^{1D}R^{1C} and —S(O)₁₋₂NR^{1D}R^{1C};
each R^{1B} is independently selected from H, C₁-C₄ alkyl, C₁-C₄ alkenyl, C₁-C₄ alkynyl, C₁-C₅ fluoroalkyl, halogen, nitro, and —CN;
each R² is independently selected from H and methyl;
each X is independently selected from a bond, —O—, and —NR^{1E}—, in which
each R^{1E} is independently selected from H and methyl;
each R³ is independently selected from —C(O)R^{1C}, —C(O)OR^{1C}, —C(O)NR^{1D}R^{1C}, in which
each R^{1C} is independently selected from H, C₁-C₄ alkyl, C₁-C₄ alkenyl, C₁-C₄ alkynyl, C₁-C₅ fluoroalkyl, C₁-C₅ hydroxyalkyl and (C₁-C₅ alkoxy)C₁-C₃ alkyl, and
each R^{1D} is independently selected from H, C₁-C₄ alkyl, C₁-C₄ alkenyl, C₁-C₄ alkynyl, C₁-C₅ fluoroalkyl, C₁-C₅ hydroxyalkyl, (C₁-C₅ alkoxy)C₁-C₃ alkyl, —S(O)₁₋₂(C₁-C₃ alkyl), —C(O)(C₁-C₃ alkyl) and —C(O)O(C₁-C₃ alkyl);
L is selected from 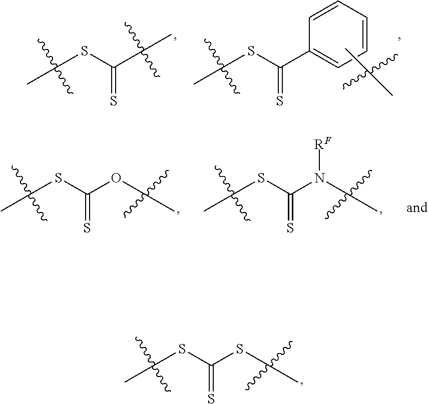
in which
each R^{F} is selected from H and C₁-C₄ alkyl; and
wherein n and m are selected such that the ratio of the molar mass in kg/mol of the segment of the surfactant having the partial structure 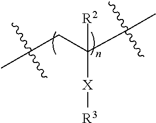
to (m+1) is from about 0.05 to about 1.

11. A mixture of the surfactants according to claim 10, wherein the dispersity of the mixture is less than about 2.5.

12. A method for preparing a formulation, comprising
providing a biologically active compound having low water solubility; and
combining the biologically active compound with a mixture according to claim 11.

13. A formulation comprising a mixture according to claim 11 and a biologically active compound having low water solubility.

14. A method for synthesizing a surfactant, comprising
performing a chain transfer polymerization reaction with an initiator, a monomer, and a chain transfer agent to provide a hydrophilic segment having repeating units, wherein
the initiator comprises a hydrophilic unit comprising less than 20 carbons; and
the chain transfer agent comprises a hydrophobic unit comprising from 6 to 40 carbons;
wherein the hydrophilic group and the hydrophobic unit are covalently attached to the hydrophilic segment; and
wherein the ratio of the molar mass of the hydrophilic segment in kg/mol to the number of carbons in the hydrophobic unit is from about 0.05 to about 1.

15. A formulation comprising
a surfactant consisting of a hydrophilic segment having repeating units and a first end and a second end;
the first end having a hydrophilic first end group; and
the second end having a second end group; wherein
the first end group comprises from 2 to 20 carbons;
the second end group comprises a linker and a hydrophobic unit, wherein the hydrophobic unit comprises from 2 to 40 carbons; and
the ratio of the molar mass of the hydrophilic segment in kg/mol to the number of carbons in the second end group is from about 0.05 to about 10, or
the surfactant according to claim 10; and
hydroxypropyl methylcellulose acetate succinate (HPMCAS).

16. A micellar composition comprising a biologically active compound having low water solubility and a mixture according to claim 11 in a polar solvent.

17. A method for preparing a solid formulation comprising a biologically active compound having low water solubility, comprising
forming a composition comprising a mixture according to claim 11, the biologically active compound, and a solvent; and
removing the solvent from the composition.

18. A solid formulation according to claim 13 prepared by
spray drying a solution comprising the biologically active compound and the mixture;
freeze drying a solution comprising the biologically active compound and the mixture; or
hot-melt extruding a mixture comprising the biologically active compound and the mixture.

19. A method for reconstituting a solid formulation prepared according to claim 18, comprising
forming a composition comprising the solid formulation and a polar solvent; and
agitating the composition.

20. A method for applying a biologically active compound having low water solubility to a substrate, comprising contacting the substrate with the formulation of claim 13.

21. A method for delivering a biologically active compound having low water solubility to an animal, comprising administering to the animal the formulation of claim 13.

22. (canceled)
